(19) United States
(12) Patent Application Publication
(10) Pub. No.: US 2024/0034722 A1
(43) Pub. Date: Feb. 1, 2024

(54) PRETARGETING IMAGING AGENTS

(71) Applicant: Eli Lilly and Company, Indianapolis, IN (US)

(72) Inventors: Aldo CAGNOLINI, West Chester, PA (US); Adam Thomas HOYE, Bala Cynwyd, PA (US); Ximin LI, Wynnewood, PA (US); Justin Patrick WRIGHT, Drexel Hill, PA (US); Hui XIONG, Chesterbrook, PA (US)

(21) Appl. No.: 18/257,368

(22) PCT Filed: Dec. 15, 2021
(86) PCT No.: PCT/US2021/063552

§ 371 Date: Jun. 14, 2023

Related U.S. Application Data
(60) Provisional application No. 63/126,100, filed on Dec. 16, 2020.
(60) Provisional application No. 63/166,484, filed on Mar. 26, 2021.

(51) Int. Cl. C07D 401/12; C07D 409/14

(52) U.S. Cl. CPC C07D 401/12; C07D 409/14

(57) ABSTRACT

The present invention provides novel compounds:

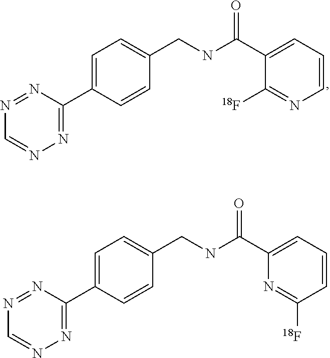 [SMILES: O=C(NCc1ccc(-c2nncnn2)cc1)c1cccnc1[18F] ; O=C(NCc1ccc(-c2nncnn2)cc1)c1cccc([18F])n1]

and methods of making such compounds, methods of using such compounds for pretargeted imaging, and preparations of formulations for such use.

The present invention relates to a compound N-(4-(1,2,4,5-tetrazin-3-yl)benzyl)-2-fluoronicotinamide, and the [¹⁸F]-radiolabelled version of this compound, and to pharmaceutically acceptable salts of these compounds, and to intermediates for preparation of these compounds, and to methods of using these compounds for pretargeted imaging, and to compositions and formulations of these compounds for diagnostic imaging (such as pretargeted imaging), and to methods of pretargeted imaging using these compounds, compositions, and formulations.

The present invention also relates to a compound N-(4-(1,2,4,5-tetrazin-3-yl)benzyl)-6-fluoropicolinamide, and the ¹⁸F-labelled version of this compound, and to pharmaceutically acceptable salts of these compounds, and to intermediates for preparation of these compounds, and to methods of using these compounds for pretargeted imaging, and to compositions and formulations of these compounds for diagnostic imaging (such as pretargeted imaging), and to methods of pretargeted imaging using these compounds, compositions, and formulations.

Historically, PET imaging using large molecules has been achieved through direct labelling of full-length antibodies. Antibodies possess exquisite specificity and selectivity but are often hindered by their slow clearance and poor brain penetration. Imaging with antibodies utilizes long-lived radionuclides where imaging is performed 7-10 days post injection of the radioimmunoconjugate to allow for the non-specific background signal to clear. This timeline is not easily incorporated into clinical practice and exposes the patient to elevated levels of radioactivity. In order to minimize disruption to normal clinical practice and radioactive exposure to the patient, pretargeted-based imaging systems have been developed. This pretargeted approach is a two-step process based on the biorthogonal inverse-electron-demand Diels-Alder (IEDDA) reaction between tetrazines and trans-cyclooctene (TCO) derivatives, which takes advantage of the specificity and selectivity of a large molecule and the rapid pharmacokinetics of small molecules with short-lived radionuclides. There are numerous preclinical examples of pretargeted imaging that have been described in the literature for peripheral targets (see J. Med. Chem. 2017, 60, 8201-8217 and J. Label Compd. Radiopharm 2014, 57 285-290.).

For antibody-based CNS imaging agents, the blood-brain barrier (BBB) presents an additional challenge. In 2017, Prof. Syvanen and coworkers (Uppsala University) demonstrated improved brain uptake of bispecific antibodies targeting Aβ protofibrils, through transferrin receptor (TfR)-mediated transport across the blood brain barrier.

Subsequent PET imaging studies using ¹²⁴I-labeled antibodies showed differentiated distribution between transgenic and wild-type mice, 3 days post injection. The distribution pattern in various brain regions were in good correlation with Aβ pathology see, Stina Syvänen et al. Theranostics, 2017; 7(2): 308-318. See also Syvänen S, Fang XT, Faresjö R, Rokka J, Lannfelt L, Olberg DE, Eriksson J, Sehlin D. Fluorine-18-Labeled Antibody Ligands for PET Imaging of Amyloid-β in Brain. ACS Chem. Neurosci. 2020, 11, 4460-4468.

Beyond brain-penetrant large molecules, the other requisite for any successful CNS pretargeted imaging studies is the availability of brain-penetrant, fast-clearing, reactive yet stable, small molecule chasers containing reactive tetrazine groups. Several ¹¹C- and ¹⁸F-labeled small molecule tetrazine chasers have been reported to have appreciable brain uptake (see, Hannes Mikula et al. Bioconjugate Chem. 2016, 27, 7, 1707-1712; Hannes Mikula et al. Angew. Chem. Int. Ed. 2014, 53, 9655-9659). However, their application in CNS pretargeted imaging studies have not been reported.

In 2019, Brendon Cook and collaborators reported the first case of CNS pretargeted imaging to study the distribution of an antisense oligonucleotide (ASO) in rat brain (2019 World Molecular Imaging Congress Conference, Poster 139). Rats were intrathecally administered 2.5 mM ASO-TCO conjugate in 30 μL saline solution and followed with an intravenous injection of a CNS-penetrant tetrazine, [¹⁸F]537-Tz, 24 and 168 hours post ASO-TCO administration. Static PET-CT scans were performed 75-90 minutes after administration of [¹⁸F]537-Tz. Higher radiotracer uptake was observed in the brain and spine of the animals that received ASO-TCO relative to the control group. It was also reported that [¹⁸F]537-Tz showed brain uptake (1.7±0.9% ID by 10 min post-injection) in wild type mice by dynamic PET imaging. This [¹⁸F]537-Tz compound is believed to be 2-(4-(1,2,4,5-tetrazin-3-yl)phenyl)-N-(2-fluoroethyl)acetamide:

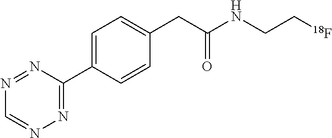 [SMILES: O=C(Cc1ccc(-c2nncnn2)cc1)NCC[18F]]

By comparison, dynamic PET imaging in CD-1 male mice of the [¹⁸F]N-(4-(1,2,4,5-tetrazin-3-yl)benzyl)-2-fluoronicotinamide and [¹⁸F]N-(4-(1,2,4,5-tetrazin-3-yl)benzyl)-6-fluoropicolinamide disclosed herein showed that these compounds readily cross the blood-brain-barrier and reaches a peak brain uptake of 3.3±0.4% ID/g and 4.3±0.3% ID/g, respectively. These compounds then displayed a steady clearance from the brain to near background levels (muscle) by 60 minutes post injection. These agents that possess robust brain penetration followed by rapid and complete washout from the brain provides a larger window to achieve higher signal to background ratios resulting in better image quality. Thus, we expect the compounds disclosed herein to provide an advantage for pretargeted CNS imaging.

The present embodiments provide novel compounds, compositions, formulations and methods for pretargeted imaging. This type of improved technology advancing the capacity to image patients is thus also needed to expand the clinical benefits and impact of diagnostic imaging. An improved imaging agent will provide enhanced pretargeted images, as compared with currently known agents.

The present embodiments also provide the compound N-(4-(1,2,4,5-tetrazin-3-yl)benzyl)-2-fluoronicotinamide, also referred to herein as “Compound 1”, which can be structurally represented as the compound of Formula I:

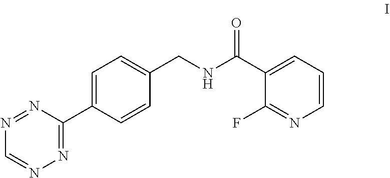 [SMILES: O=C(NCc1ccc(-c2nncnn2)cc1)c1cccnc1F]

The compound of Formula I is shown above as a free base. The compound of Formula I may also be converted into a pharmaceutically acceptable salt and used in the salt form in the present embodiments.

The present embodiments provide the compound ¹⁸F-version of Compound 1, which is also referred to herein as “Compound 2”, which can be structurally represented as the compound of Formula II:

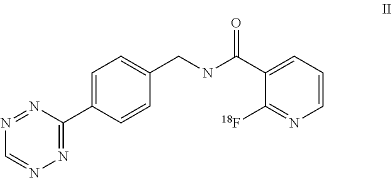 [SMILES: O=C(NCc1ccc(-c2nncnn2)cc1)c1cccnc1[18F]]

The compound of Formula II is shown above as a free base. The compound of Formula II may also be converted into a pharmaceutically acceptable salt and used in the salt form in the present embodiments.

The present embodiments provide for use of pharmaceutically acceptable salts of either Formula I or Formula II or the use of the compounds as the free base.

The present embodiments further provide the use of the compound of Formula I and/or the compound of Formula II, and/or mixtures thereof, for the preparation of imaging agents, such as, for example, pretargeted imaging agents.

The present embodiments provide for the use of compounds of Formula I or II, for the manufacture of a radiopharmaceutical agent for imaging (pretargeted imaging) in humans. In another aspect the invention provides methods of preparing compounds of Formula I or II.

In another aspect the present embodiments provide a pharmaceutical composition comprising Compound 1 or Compound 2, or pharmaceutically acceptable salt thereof, which is formulated in ethanol (such as, for example 10% EtOH (v/v)) and buffer (which may be PBS buffer), preferably for use in humans. It should also be noted that, in some embodiments, the formulation does not include ascorbate or ascorbic acid, as it has been found that the tetrazine moiety in Formula I and Formula II could be readily reduced by ascorbate formulation. Thus, although ascorbate formulation is readily used in many formulations for imaging, it may not be suitable for imaging with the compounds of Formula I or Formula II.

The present invention also provides methods for pretargeted imaging comprising introducing into a patient a detectable quantity of Compound 1 or 2, or pharmaceutically acceptable salt thereof, or a composition thereof.

The present embodiments also provide the N-(4-(1,2,4,5-tetrazin-3-yl)benzyl)-6-fluoropicolinamide, also referred to herein as “Compound 3”, which can be structurally represented as the compound of Formula III:

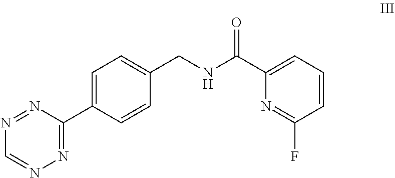 [SMILES: O=C(NCc1ccc(-c2nncnn2)cc1)c1cccc(F)n1]

The compound of Formula III is shown as a free base. The compound of Formula III may also be converted into a pharmaceutically acceptable salt and used in the salt form in the present embodiments.

The present embodiments provide the compound ¹⁸F-version of Compound 3, which is also referred to herein as “Compound 4”, which can be structurally represented as the compound of Formula IV:

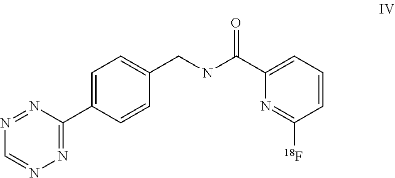 [SMILES: O=C(NCc1ccc(-c2nncnn2)cc1)c1cccc([18F])n1]

The compound of Formula IV is shown as a free base. The compound of Formula IV may also be converted into a pharmaceutically acceptable salt and used in the salt form in the present embodiments.

The present embodiments provide for the use of pharmaceutically acceptable salts of either Formula III or IV or the use of the compounds as the free base.

The present embodiments further provide the use of the compound of Formula III and/or the compound of Formula IV, and/or mixtures thereof, for the preparation of imaging agents, such as, for example, pretargeted imaging agents.

The present embodiments provide for the use of compounds of Formula III or IV, for the manufacture of a radiopharmaceutical agent for imaging (pretargeted imaging) in humans. In another aspect the invention provides methods of preparing compounds of Formula III or IV.

In another aspect the present embodiments provide a pharmaceutical composition comprising Compound 3 or Compound 4, or pharmaceutically acceptable salt thereof, which is formulated in ethanol (such as, for example 10% EtOH (v/v)) and buffer (which may be PBS buffer), preferably for use in humans. It should also be noted that, in some embodiments, the formulation does not include ascorbate or ascorbic acid, as it has been found that the tetrazine moiety in Formula III and Formula IV could be readily reduced by ascorbate formulation. Thus, although ascorbate formulation is readily used in many formulations for imaging, it may not be suitable for imaging with the compounds of Formula III or Formula IV.

The present invention also provides methods for pretargeted imaging comprising introducing into a patient a detectable quantity of Compound 3 or 4, or pharmaceutically acceptable salt thereof, or a composition thereof.

The present embodiments provide the compound of Formula V, which can be structurally represented below:

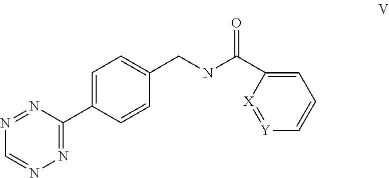

wherein, X may be C—F, C-¹⁸F, or N
   Y may be C—F, C-¹⁸F, or N
   wherein one of (but not both) X and Y is N,
   wherein, when X is N, Y is C—F or C-¹⁸F;
   and wherein when Y is N, X is C—F or C-¹⁸F, or pharmaceutically acceptable salts thereof.

Those skilled in the art will appreciate that Formula V represents as genus that covers the Compounds 1, 2, 3 and 4 above (as well as pharmaceutically acceptable salts of these compounds). Thus, the above-recited disclosure with respect to the embodiments of Compounds 1, 2, 3 and 4 (or Formulas I-IV), as well as their associated uses, compositions, formulations, etc, apply equally to the compounds of Formula V.

The present embodiments provide for the use of pharmaceutically acceptable salts of either Formula V or the use of the compounds as the free base.

The present embodiments further provide the use of the compounds of Formula V, and/or mixtures thereof, for the preparation of imaging agents, such as, for example, pretargeted imaging agents.

The present embodiments provide for the use of compounds of Formula V, for the manufacture of a radiopharmaceutical agent for imaging (pretargeted imaging) in humans. In another aspect the invention provides methods of preparing compounds of Formula V.

In another aspect the present embodiments provide a pharmaceutical composition comprising the compounds of Formula V, or a pharmaceutically acceptable salt thereof, which is formulated in ethanol (such as, for example 10% EtOH (v/v)) and buffer (which may be PBS buffer), preferably for use in humans. It should also be noted that, in some embodiments, the formulation does not include ascorbate or ascorbic acid, as it has been found that the tetrazine moiety in Formula V could be readily reduced by ascorbate formulation. Thus, although ascorbate formulation is readily used in many formulations for imaging, it may not be suitable for imaging with the compounds of Formula V.

The present invention also provides methods for pretargeted imaging comprising introducing into a patient a detectable quantity of compounds of Formula V, or pharmaceutically acceptable salt thereof, or a composition thereof.

The following Preparations, and Examples are provided to better elucidate the practice of the present invention. Suitable reaction conditions for the steps of these Schemes, Preparations, and Examples are well known in the art and appropriate modification of reaction conditions, including substitution of solvents and co-reagents are within the ability of the skilled artisan.

Furthermore, the skilled artisan will appreciate that in some circumstances, the order in which moieties are introduced is not critical. The particular order of steps required to produce the compounds of Formula I or Formula II or Formula III or Formula IV is dependent upon the particular compound being synthesized, the starting compound, and the relative lability of the substituted moieties, as is well appreciated by the skilled chemist. These compounds may be protected or modified at a convenient point in the synthesis by methods well known in the art. The intermediates and final products of the present invention may be further purified, if desired by common techniques such as recrystallization or chromatography over solid supports such as silica gel or alumina.

The compounds of the present invention are preferably formulated as radiopharmaceutical compositions administered by a variety of routes. Preferably, such compositions are for intravenous use, preferably in humans. Such pharmaceutical compositions and processes for preparing same are well known in the art. See, e.g., Remington: The Science and Practice of Pharmacy (P.P. Gerbino, 21^{st} ed., Lippincott Williams & Wilkins, 2006).

Preferred formulations may be preparations of Compound 1 or Compound 2. Preferred formulations may be preparations of Compound 3 or Compound 4. Particularly preferred is Compound 1 or Compound 2 or Compound 3 or Compound 4 prepared according to the procedures described herein. A preferred formulation of Compound 1 or Compound 2 is formulated in ethanol, such as for example, 10% EtOH (v/v). This formulation may also include a buffer, such as a PBS buffer. Other ingredients may also be used. A preferred formulation of Compound 3 or Compound 4 is formulated in ethanol, such as for example, 10% EtOH (v/v). This formulation may also include a buffer, such as a PBS buffer. Other ingredients may also be used.

Compounds of Formula I and II and III and IV have been discovered to be surprisingly and unexpectedly advantageous for pretargeted imaging, preferably including human clinical imaging. In some embodiments, the compounds of Formula I, II, III, IV, or V may be used for pretargeted imaging. For example, the compounds of Formulas I-V may be brain penetrant, and thus may be used as a tracer for CNS (central nervous system) pretargeted imaging. In some embodiments, the pretargeted imaging of CNS targets may be achieved in 3 or 4 steps:
1. I.V. (intravenous) or I.T (intrathecal) administration of biologics-TCO conjugate (for example, shuttled bispecific antibody-TCO conjugate, or oligonucleotide-TCO conjugate);
2. Wait for sufficient time (days) to allow distribution and systematic clearance of biologics-TCO conjugate
3. An optional step of I.V. injection of periphery-restricted tetrazines to mask peripherally circulating biologics-TCO conjugate;
4. I.V. administration of a brain penetrant compound of Formula I or Formula II or Formula III or Formula IV of Formula V, followed by brain PET imaging.

With respect to the preparation of biologics-TCO conjugate, the use of such technology is published in the literature (see below) and is known for oncology pretargeted imaging studies):
1. Bioconjugate Chem. 2018, 29, 538-545 (TCO antibody conjugation; Use of clearing agent to mask circulating antibody-TCO)
2. Bioconjugate Chem. 2013, 24, 1210-1217 (TCO antibody conjugation) J. Med. Chem. 2017, 60, 8201-8217 (TCO antibody conjugation, Pretargeting Radioligand Optimization)

For oligonucleotide-TCO conjugation, this type of conjugate (a TCO-PEG4 Oligo Modification) is commercially available from the Bio-Synthesis company of Lewisville, Texas, USA. Thus, those skilled in the art will appreciate how to accomplish the preparation of the biologics-TCO conjugate.

Some of the potential benefits of pretargeted imaging include: the ability to use short-lived radionuclides by separating the delivery of the radioactivity from the targeting vector such as biologics with high specificity and selectivity and the ability to reduce patient exposure to radioactivity.

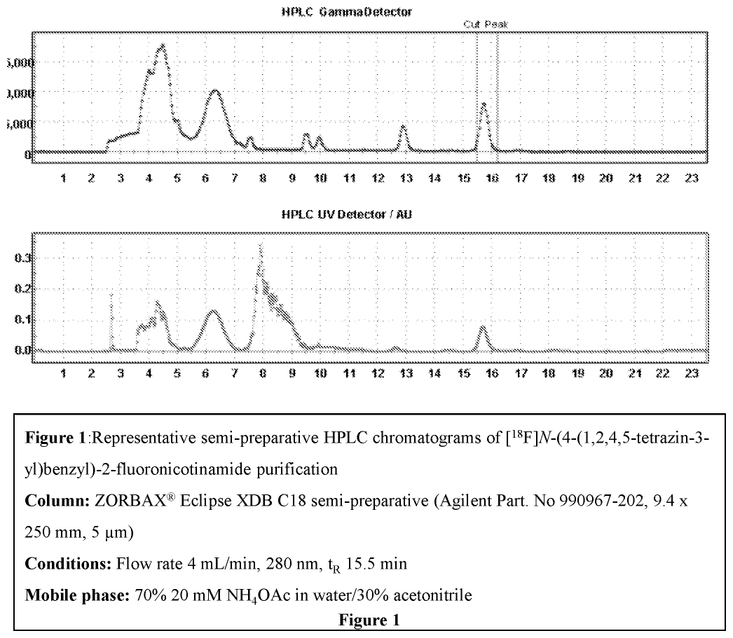

FIG. 1 is a representative semi-preparative HPLC chromatograms of [¹⁸F]N-(4-(1,2,4,5-tetrazin-3-yl)benzyl)-2-fluoronicotinamide purification;

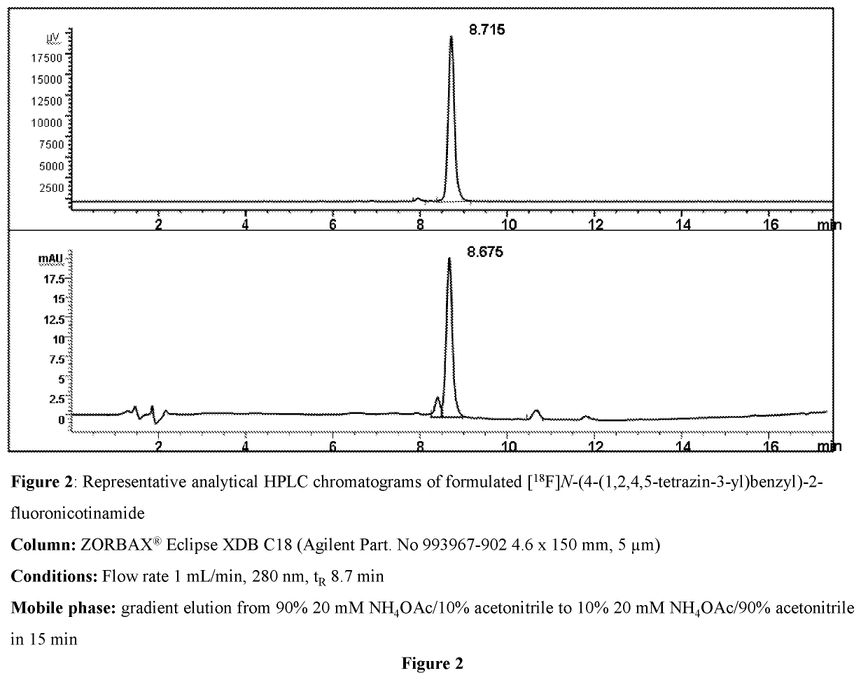

FIG. 2 is a representative analytical HPLC chromatograms of formulated [¹⁸F]N-(4-(1,2,4,5-tetrazin-3-yl)benzyl)-2-fluoronicotinamide;

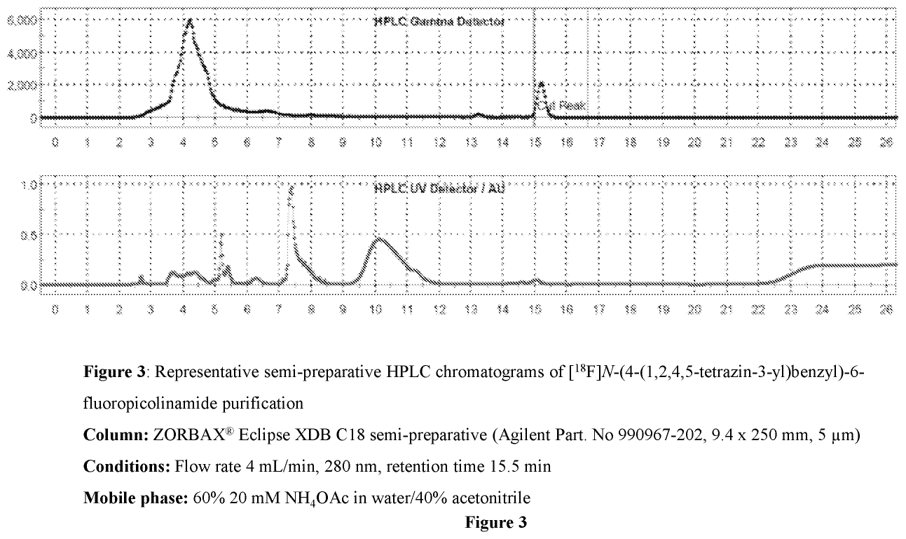

FIG. 3 is a representative semi-preparative HPLC chromatograms of [¹⁸F]N-(4-(1,2,4,5-tetrazin-3-yl)benzyl)-6-fluoropicolinamide purification;

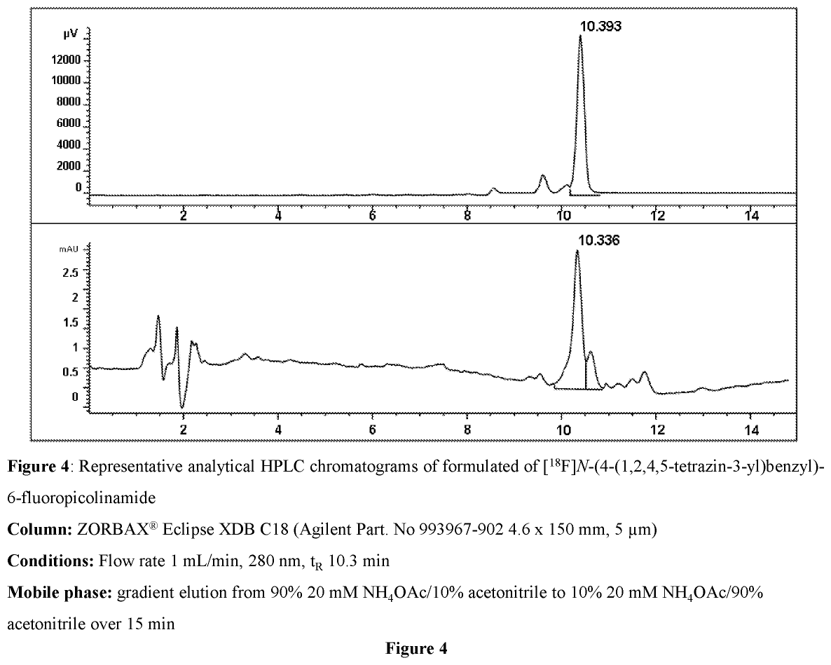

FIG. 4 is a representative analytical HPLC chromatograms of formulated of [¹⁸F]N-(4-(1,2,4,5-tetrazin-3-yl)benzyl)-6-fluoropicolinamide;

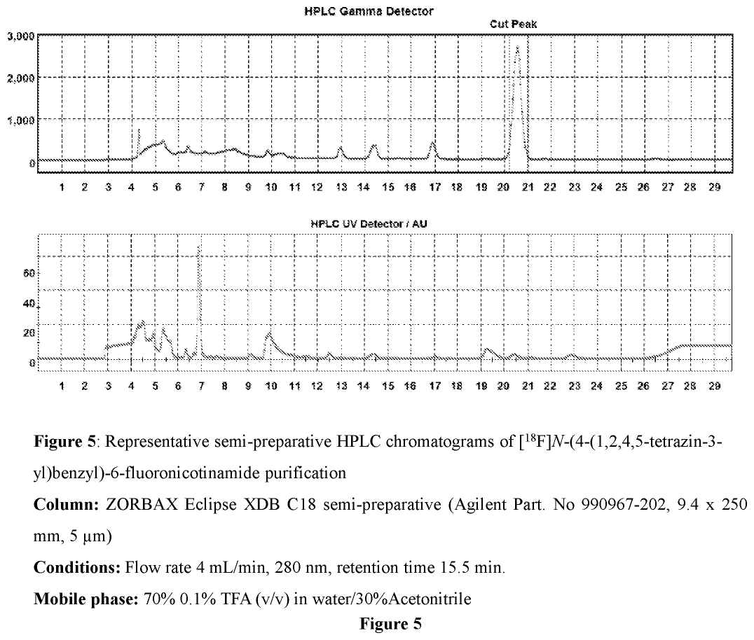

FIG. 5 is a representative semi-preparative HPLC chromatograms of [¹⁸F]N-(4-(1,2,4,5-tetrazin-3-yl)benzyl)-6-fluoronicotinamide purification;

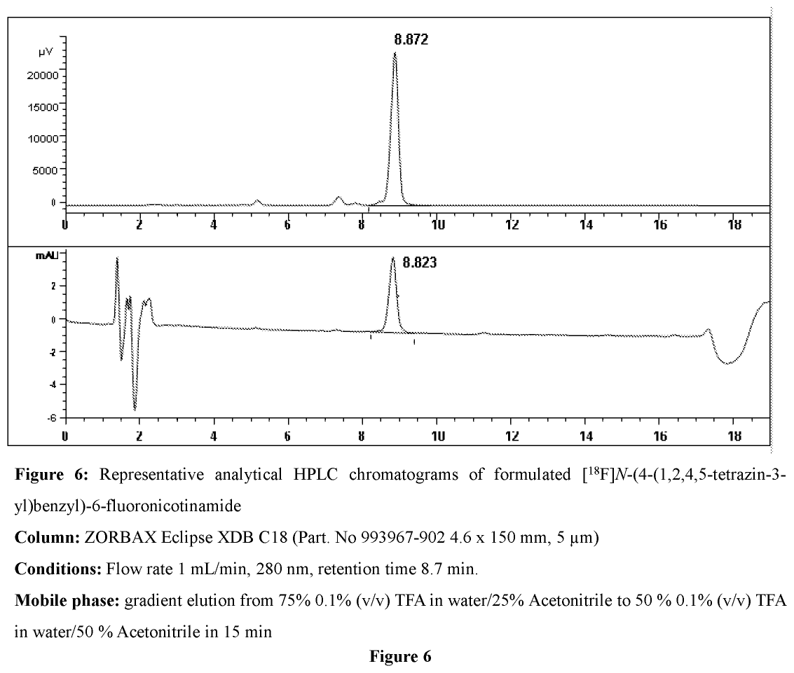

FIG. 6 is a representative analytical HPLC chromatograms of formulated [¹⁸F]N-(4-(1,2,4,5-tetrazin-3-yl)benzyl)-6-fluoronicotinamide;

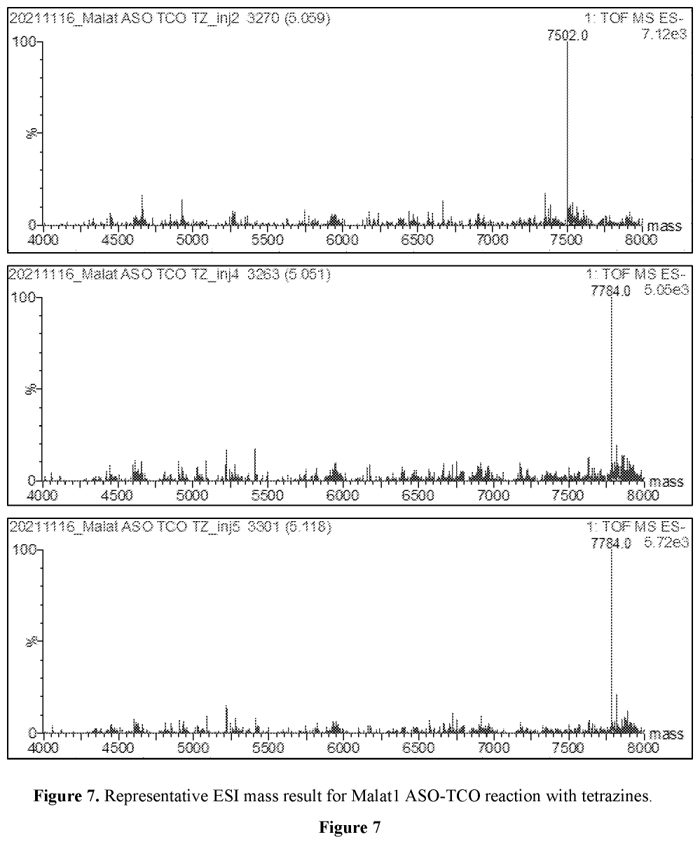

FIG. 7. Representative ESI mass result for Malat1 ASO-TCO reaction with tetrazines; and

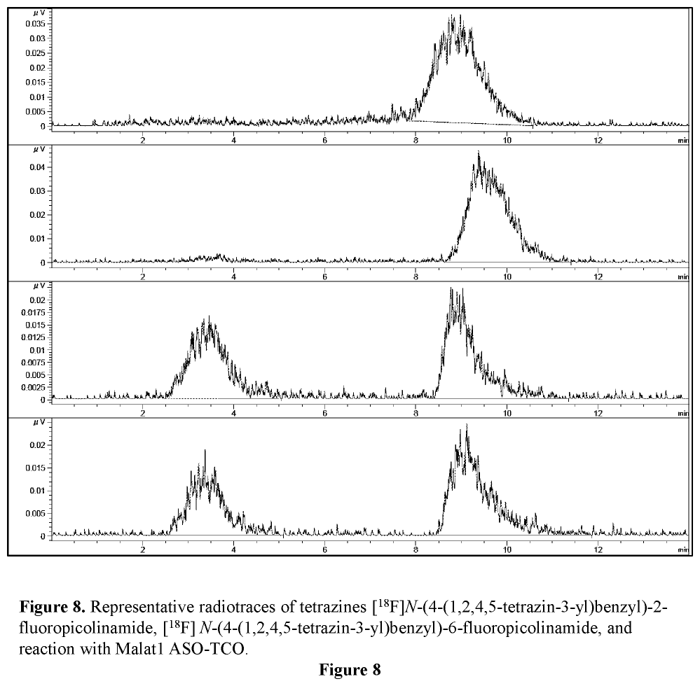

FIG. 8. Representative radiotraces of tetrazines [¹⁸F]N-(4-(1,2,4,5-tetrazin-3-yl)benzyl)-2-fluoropicolinamide, [¹⁸F] N-(4-(1,2,4,5-tetrazin-3-yl)benzyl)-6-fluoropicolinamide, and reaction with Malat1 ASO-TCO.

Certain abbreviations may be used below. These abbreviations mean as follows: “CAS #” refers to Chemical Abstracts Registry number; “Ci” refers to Curie or Curies; “CT” refers to computed tomography; “δ” refers to chemical shift in nuclear magnetic resonance spectroscopy; “DMF” refers to N,N-dimethylformamide; “DMSO” refers to dimethyl sulfoxide; “DMSO-d₆” refers to deuterated DMSO; “ES/MS” refers to electrospray-mass spectrometry; “EtOH” refers to ethanol or ethyl alcohol; ‘HATU” refers to 1-[bis(dimethylamino)methylene]-1H-1,2,3-triazolo[4,5-b]pyridinium 3-oxide hexafluorophosphate; “HPLC” refers to High Performance Liquid Chromatography; “h” and “hr” refer to hour or hours; “min” refers to minute or minutes; “% ID/g” refers to % injected dose per gram; “mCi” refers to milliCurie or milliCuries; “MHz” refers to megahertz; “μL” refers to microliter or microliters; “N” refers to number of replications or sample size in an experiment; “NMR” refers to nuclear magnetic resonance; “PET” refers to positron emission tomography” “OAc” refers to acetate; “ppm” refers to parts per million; “s” refers to singlet; “SEM” refers to standard error of the mean; “t_{R}” refers to retention time; “THF” refers to tetrahydrofuran.

General Chemistry and Preparations

The following Preparations and Examples further illustrate the invention and represent typical syntheses of the compounds of the invention. The reagents and starting materials are readily available or may be readily synthesized by one of ordinary skill in the art. It should be understood that the Preparations and Examples are set forth by way of illustration and not limitation, and that various modifications may be made by one of ordinary skill in the art.

NMR spectroscopy for ¹H and ¹⁹F spectra are performed on a Bruker Avance™ III HD 400 MHz NMR spectrometer, obtained as CDCl₃ or DMSO-d₆ solutions reported in ppm using residual solvent resonances (CDCl₃, 7.26 ppm; DMSO-d₆, 2.50 ppm) as ¹H NMR reference standards. No reference standard is used for ¹⁹F spectra. When peak multiplicities are reported, the following abbreviations may be used: s (singlet), d (doublet), t (triplet), q (quartet), m (multiplet), br s (broad singlet), dd (doublet of doublets), dt (doublet of triplets). Coupling constants (J), when reported, are reported in hertz (Hz).

ES/MS is performed on a Waters® Acquity UPLC system. Electrospray mass spectrometry measurements (acquired in positive and/or negative mode) are performed on a Waters® Acquity QDa mass detector interfaced to the UPLC system. LC-MS conditions are as follows unless otherwise noted in the experimental sections below. Column: Waters Acquity UPLC® BEH 2.1×30 mm, 1.7 μ; wavelength 250-650 nm. Using one of two gradients: gradient 1: initial hold at 10% B for 0.5 min, 10-98% B in 3.5 min, hold 98% B for 0.5 min, and return to 10% B for 0.6 min; gradient 2: initial hold at 10% B for 0.5 min, 10-98% B in 1.5 min, hold 98% B for 0.5 min, and return to 10% B to re-equilibrate; column temperature: 40° C. +/−10° C.; flow rate: 0.5 mL/min; solvent for A: deionized water with 0.1% HCOOH; solvent for B: 100% acetonitrile.

Of course, other instruments and means of detecting and for ES/MS are known in the art and would be known by a person of ordinary skill.

Preparative and analytical HPLC conditions, when used, are detailed below.

### EXAMPLE 1

### N-(4-(1,2,4,5-tetrazin-3-yl)benzyl)-2-fluoronicotinamide

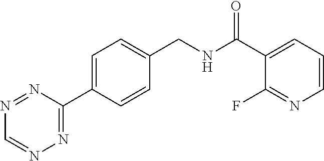 [SMILES: O=C(NCc1ccc(-c2nncnn2)cc1)c1cccnc1F]

To a stirred solution of [4-(1,2,4,5-tetrazin-3-yl)phenyl]methanamine hydrochloride (115 mg, 0.51 mmol, Click Chemistry Tools, CAS #1416711-59-5) and 2-fluoropyridine-3-carboxylic acid (90 mg, 0.64 mmol) in dichloromethane (6 mL) is added HATU (260 mg, 0.67 mmol) and N,N-diisopropylethylamine (0.2 mL, 1.0 mmol). The reaction mixture is stirred at room temperature overnight. The reaction mixture is diluted with dichloromethane, the organic layer is washed sequentially with saturated aqueous NaHCO₃, saturated aqueous NaCl, dried over Na₂SO₄, filtered, and concentrated under reduced pressure. The resulting residue is purified by column chromatography over silica, eluting with a gradient of 0-50% dichloromethane in ethyl acetate, to give the title compound as a purple solid after solvent evaporation of the desired chromatographic fractions (150 mg, 64% yield, containing hexafluorophosphate salt). The title compound is further purified (75 mg, 0.16 mmol) by dissolving in 20 mL dichloromethane and washing with 3×0.5 M aqueous potassium acetate solution. The resulting organic layer is dried over Na₂SO₄, filtered, and concentrated under reduced pressure to afford the purified title compound as a purple solid (51 mg). ¹H NMR (400.13 MHz, DMSO-d₆) δ ppm: 4.63 (d, J=6.0 Hz, 2H), 7.51-7.47 (m, 1H), 7.65 (d, J=8.6 Hz, 2H), 8.26-8.22 (m, 1H), 8.36-8.39 (m, 1H), 8.51-8.48 (m, 2H), 9.17-9.12 (m, 1H), 10.58 (s, 1H). ¹⁹F NMR (376.45 MHz, DMSO-d₆) δ ppm:−67.6. ES/MS (m/z): 311 (M+H).

### Preparation 1

### 2-[2-(3,5-dimethoxyphenyl)-4-methyl-phenyl]sulfanylpyridine-3-carboxylic acid

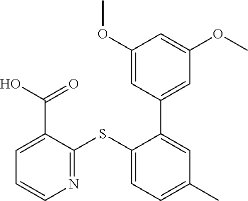 [SMILES: COc1cc(OC)cc(-c2cc(C)ccc2Sc2ncccc2C(=O)O)c1]

A solution of methyl 2-chloropyridine-3-carboxylate (380 mg, 2.2 mmol) and 2-ethylhexyl 3-[2-(3,5-dimethoxyphenyl)-4-methyl-phenyl]sulfanylpropanoate (900 mg, 2.0 mmol), tris(dibenzylideneacetone)dipalladium(0) (180 mg, 0.20 mmol) and 4,5-bis(diphenylphosphino)-9,9-dimethylxanthene (230 mg, 0.40 mmol) in 1,4-dixoane (8 mL) is bubbled with N₂ for 5 minutes, and a 1M solution of potassium tert-butoxide in THF (2.20 mL, 2.20 mmol) is added. The reaction mixture is sealed and heated to 100° C. overnight. The reaction mixture is cooled to room temperature and 1N aqueous NaOH (6 mL) is added. The resulting mixture is stirred at room temperature for 2-3 hr, diluted with water (30 mL), and extracted with ethyl acetate (2×30 mL). The aqueous layer is neutralized to pH ˜1 with 1N aqueous HCl and extracted with ethyl acetate (2×30 mL). The organic layers are combined, washed with saturated aqueous NaCl, dried over Na₂SO₄, filtered, and concentrated under reduced pressure. The resulting residue is purified by column chromatography over silica, eluting with a gradient of 0-100% hexanes in ethyl acetate, to afford the title compound (270 mg, 33% yield) as a yellow solid. ES/MS (m/z): 382 [M+H].

### Preparation 2

### 2-[2-(3,5-dimethoxyphenyl)-4-methyl-phenyl]sulfinyl-N-[[4-(1,2,4,5-tetrazin-3-yl)phenyl]methyl]pyridine-3-carboxamide

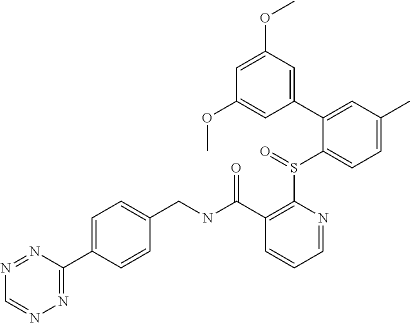 [SMILES: COc1cc(OC)cc(-c2cc(C)ccc2S(=O)c2ncccc2C(=O)NCc2ccc(-c3nncnn3)cc2)c1]

To a stirred mixture of 2-[2-(3,5-dimethoxyphenyl)-4-methyl-phenyl]sulfanylpyridine-3-carboxylic acid (100 mg, 0.26 mmol) and [4-(1,2,4,5-tetrazin-3-yl)phenyl]methanamine hydrochloride (59 mg, 0.26 mmol, Click Chemistry Tools, CAS #1416711-59-5) and HATU (122 mg, 0.41 mmol) in dichloromethane (2.6 mL) is added N,N-diisopropylethylamine (120 μL, 0.67 mmol). The reaction mixture is stirred at room temperature overnight. The volatiles are removed under reduced pressure. The resulting residue is dissolved in ethyl acetate (50 mL) and washed sequentially with 0.5 M aqueous KHCO₃ (2×30 mL) and saturated aqueous NaCl (30 mL). The organic extracts are dried over Na₂SO₄, filtered, and concentrated under reduced pressure. The resulting residue is purified by chromatography on silica gel (0-100% hexane/ethyl acetate) to afford 2-[2-(3,5-dimethoxyphenyl)-4-methyl-phenyl]sulfanyl-N-[[4-(1,2,4,5-tetrazin-3-yl)phenyl]methyl]pyridine-3-carboxamide (121 mg, 84% yield) as a pink foam.

To a stirred solution of 2-[2-(3,5-dimethoxyphenyl)-4-methyl-phenyl]sulfanyl-N-[[4-(1,2,4,5-tetrazin-3-yl)phenyl]methyl]pyridine-3-carboxamide (121 mg, 0.22 mmol) in dichloromethane (2.5 mL) is added 3-chloroperoxybenzoic acid (77% pure, 36 mg, 0.16 mmol). The mixture is stirred at room temperature for 30 minutes and concentrated under reduced pressure. The resulting residue is purified by chromatography on silica gel, eluting with a gradient of 0-100% hexanes/ethyl acetate, to afford 2-[2-(3,5-dimethoxyphenyl)-4-methyl-phenyl]sulfinyl-N-[[4-(1,2,4,5-tetrazin-3-yl)phenyl]methyl]pyridine-3-carboxamide (91 mg, 0.16 mmol, 73% yield) as a pink foam: ¹H NMR (400.13 MHz, DMSO-d₆) δ ppm: 2.39 (s, 3H), 3.69 (s, 6H), 4.60-4.42 (m, 2H), 6.50-6.46 (m, 3H), 7.19 (d, J=1.1 Hz, 1H), 7.36-7.33 (m, 1H), 7.59-7.56 (m, 5H), 8.10-8.07 (m, 1H), 8.43 (d, J=8.5, 2H), 8.69-8.67 (m, 1H), 9.08 (t, 1H), 10.56 (s, 1H). ES/MS (m/z): 567 (M+H).

### Preparation 3

### 2-(2,4-dimethoxy-8-methyl-dibenzothiophen-5-ium-5-yl)-N-[[4-(1,2,4,5-tetrazin-3-yl)phenyl]methyl]pyridine-3-carboxamide trifluoromethanesulfonate

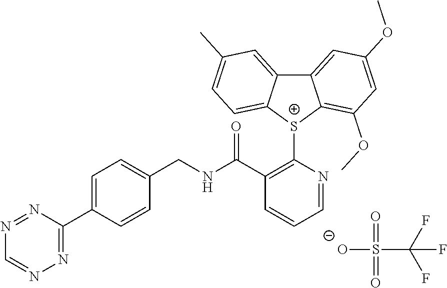 [SMILES: COc1cc(OC)c2c(c1)c1cc(C)ccc1[s+]2-c1ncccc1C(=O)NCc1ccc(-c2nncnn2)cc1 ; O=S(=O)([O-])C(F)(F)F]

To a stirred slurry of 2-[2-(3,5-dimethoxyphenyl)-4-methyl-phenyl]sulfinyl-N-[[4-(1,2,4,5-tetrazin-3-yl)phenyl]methyl]pyridine-3-carboxamide (25 mg, 0.04 mmol) and diethylaminopolystyrene (3.2 mmol/g, 100 mg, 0.32 mmol) in dichloromethane (3 mL) cooled to 0° C. is added a 1M solution of trifluoromethanesulfonic anhydride in dichloromethane (150 μL, 0.15 mmol). The reaction mixture is warmed to room temperature, stirred for 30 minutes, and the reaction is quenched by adding a drop of water. The mixture is filtered and solids are washed with dichloromethane. The combined filtrates are concentrated under reduced pressure and the resulting residue was purified by chromatography on silica gel, using a gradient of 0-15% methanol in dichloromethane, to afford the title compound (10 mg, 0.014 mmol, 32% yield) as a purple solid: ¹H NMR (400.13 MHz, DMSO-d₆) δ ppm: 2.46 (s, 3H), 3.86 (s, 3H), 4.03 (s, 3H), 4.94-4.81 (m, 2H), 6.43 (d, J=2.1 Hz, 1H), 7.45-7.42 (m, 1H), 7.67 (d, J=2.1 Hz, 1H), 7.80 (d, 8.5 Hz, 2H), 7.91-7.88 (m, 1H), 8.18 (d, J=8.4 Hz, 1H), 8.28 (s, 1H), 8.65 (d, J=8.4 Hz, 2H), 8.60-8.59 (m, 1H), 8.73-8.70 (m, 1H), 10.30 (t, 1H), 10.61 (s, 1H). ¹⁹F NMR (376.45 MHz, DMSO-d₆) δ ppm:−77.7. ES/MS (m/z): 549 (M′).

### EXAMPLE 2

### [¹⁸F]N-(4-(1,2,4,5-tetrazin-3-yl)benzyl)-2-fluoronicotinamide

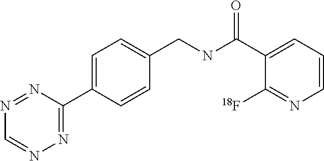 [SMILES: O=C(NCc1ccc(-c2nncnn2)cc1)c1cccnc1[18F]]

Typical radiochemical yield of the title compound radiosynthesis is 3.6±1.34% (N=3) using 0.435 to 1.442 Ci starting activity with synthesis time of 50 minutes.

[¹⁸F] Fluoride activity (0.435 to 1.442 Ci) is retained on a Sep-Pak Accell Plus QMA Plus Light Cartridge (130 mg, 37-55 μm, Waters Part #WAT023525; preconditioned with 5 mL of water for injection) and is eluted into a TRACERlab FX_{F-N} reaction vessel using 0.8 mL of an aqueous tetraethylammonium bicarbonate solution containing acetonitrile [tetraethylammonium bicarbonate (3.5 mg) in water (0.2 mL) and acetonitrile (0.6 mL)]. The eluted activity is heated to 70° C. and dried under a compressed nitrogen gas purge and vacuum for 5 minutes. The temperature is then raised to 100° C. and held for 5 minutes under vacuum yielding [¹⁸F]tetraethylammonium fluoride. The reactor is cooled to 35° C. and a solution of 2-(2,4-dimethoxy-8-methyl-dibenzothiophen-5-ium-5-yl)-N-[[4-(1,2,4,5-tetrazin-3-yl)phenyl]methyl]pyridine-3-carboxamide trifluoromethanesulfonate (1 mg, 1.43 μmol) in anhydrous DMSO (1 mL) is added. The temperature is raised to 40° C. and the reaction mixture is kept at 40° C. for 5 minutes. The mixure is then diluted with 3.5 mL of 0.1% (v/v) trifluoroacetic acid in water and the resulting crude reaction mixture loaded onto a semi-preparative HPLC column for purification (conditions listed in FIG. 1). The HPLC fraction containing the purified [¹⁸F]N-(4-(1,2,4,5-tetrazin-3-yl)benzyl)-2-fluoronicotinamide (FIG. 1) is collected into a vial containing 40 mL of water and loaded onto a Sep-Pak® Light 1cc Vac C18 cartridge (50 mg, 55-105 μm, Waters Part #WAT054955; preconditioned sequentially with 5 mL of EtOH and 5 mL of water). The retained [¹⁸F]N-(4-(1,2,4,5-tetrazin-3-yl)benzyl)-2-fluoronicotinamide is eluted using 0.5 mL of EtOH and reconstituted into a formulation of 5 mL of 10% (v/v) EtOH in phosphate-buffered saline. The amount of [¹⁸F]N-(4-(1,2,4,5-tetrazin-3-yl)benzyl)-2-fluoronicotinamide obtained ranged from 0.014 to 0.033 Ci. Representative analytical HPLC of formulated [¹⁸F]N-(4-(1,2,4,5-tetrazin-3-yl)benzyl)-2-fluoronicotinamide are shown in FIG. 2.

### EXAMPLE 3

### N-(4-(1,2,4,5-tetrazin-3-yl)benzyl)-6-fluoropicolinamide

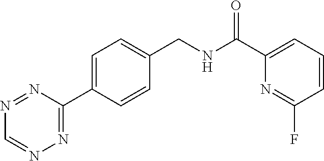 [SMILES: O=C(NCc1ccc(-c2nncnn2)cc1)c1cccc(F)n1]

To a mixture of [4-(1,2,4,5-tetrazin-3-yl)phenyl]methanamine hydrochloride (60 mg, 0.27 mmol, Click Chemistry Tools, CAS #1416711-59-5) and 6-fluoropyridine-2-carboxylic acid (45 mg, 0.32 mmol) in dichloromethane (4 mL) is added N,N-diisopropylethylamine (150 μL, 0.86 mmol) followed by HATU (135 mg, 0.35 mmol), and the reaction mixture is stirred at room temperature overnight. The resulting pink slurry is partitioned between dichloromethane and 1M aqueous citric acid. The layers are separated, and the aqueous phase is extracted with dichloromethane (3×10 mL). The combined organic extracts are dried over sodium sulfate, filtered, and concentrated under reduced pressure onto silica gel. The resulting residue is purified by chromatography on silica gel, eluting with a gradient of dichloromethane/ethyl acetate as eluents, to obtain the crude title compound contaminated with fluorophosphate salts. The crude product is dissolved in dichloromethane (10 mL) and washed with 0.5 M aqueous potassium acetate (2×10 mL). The organic extracts are dried over sodium sulfate, filtered, and concentrated to afford pure title compound (64 mg, 77% yield) as a magenta solid: ¹H NMR (400.13 MHz, DMSO-d₆) δ ppm: 4.62 (d, J=6.2 Hz, 2H), 7.45-7.43 (m, 1H), 7.61 (d, J=8.8 Hz, 2H), 8.02-7.99 (m, 1H), 8.20 (q, 1H), 8.46 (d, J=8.5 Hz, 2H), 9.39 (t, 1H), 10.56 (s, 1 H). ¹⁹F NMR (376.45 MHz, DMSO-d₆) δ ppm:−68.0. ES/MS (m/z): 311 (M+H).

### Preparation 4

### 6-[2-(3,5-dimethoxyphenyl)-4-methyl-phenyl]sulfanylpyridine-2-carboxylic acid

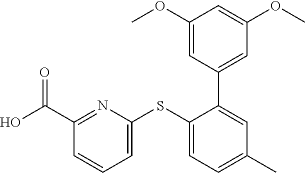 [SMILES: COc1cc(OC)cc(-c2cc(C)ccc2Sc2cccc(C(=O)O)n2)c1]

To a solution of tris(dibenzylideneacetone)dipalladium(0) (312 mg, 0.33 mmol) and bis(2-diphenylphosphinophenyl)ether (365 mg, 0.66 mmol) in N,N-dimethylacetamide (15 mL) is added a solution of methyl 6-bromopyridine-2-carboxylate (1.71 g, 8.0 mmol), 2-ethylhexyl 3-[2-(3,5-dimethoxyphenyl)-4-methyl-phenyl]sulfanylpropanoate (3.0 g, 6.6 mmol) and potassium tert-butoxide (2.3 g, 19.8 mmol) in N,N-dimethylacetamide (20 mL). The mixture is stirred at 120° C. for 6 h under N2. and combined with a smaller scale reaction (using 2-ethylhexyl 3-[2-(3,5-dimethoxyphenyl)-4-methyl-phenyl]sulfanylpropanoate (100 mg, 0.22 mmol) and methyl 6-bromopyridine-2-carboxylate (58 mg, 0.27 mmol) in N,N-dimethylacetamide). The reaction mixture is treated with aqueous 2M NaOH (10 mL) and stirred at 40° C. for 1 h. The mixture is diluted with water (70 mL), extracted with EtOAc (30 mL), and the organic layer is discarded. The aqueous phase is adjusted to pH ˜3 by addition of 1N aqueous HCl solution. The acidified mixture is extracted with EtOAc (2×30 mL). The combined organic extracts are dried over Na₂SO₄ and concentrated under reduced pressure. The resulting residue is purified by preparative-HPLC (column: YMC-Triart C18, 250×50 mm, 7 m), eluting with a gradient of 40%-80% acetonitrile in water containing 0.225% formic acid over 25 min. The desired fractions are lyophilized to give the title compound (1.49 g, 53% yield) as a yellow solid. ES/MS (m/z): 382 [M+H]. ES/MS conditions: LC (low pH): ES/MS is performed on a Shimadzu LCMS 2020 liquid chromatography system. Electrospray mass spectrometry measurements (acquired in positive mode) are performed on Scan Mode quadrupole mass spectrometer interfaced to the LC system. LC-MS column: Xtimate® C18 2.1×30 mm, 3 m, gradient: 10-80% B in 3 min, 80% B for 0.5 min, column temperature: 50° C., flow rate: 1.2 mL/min, solvent A: deionized water with 0.037% formic acid, solvent B: acetonitrile with 0.018% formic acid, wavelength UV 220 nm and 254 nm.

### Preparation 5

### 6-[2-(3,5-dimethoxyphenyl)-4-methyl-phenyl]sulfinyl-N-[[4-(1,2,4,5-tetrazin-3-yl)phenyl]methyl] pyridine-2-carboxamide

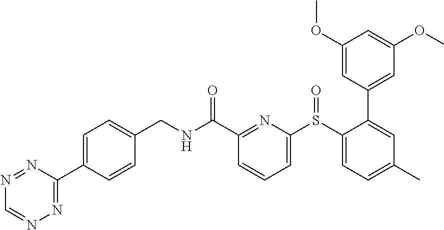 [SMILES: COc1cc(OC)cc(-c2cc(C)ccc2S(=O)c2cccc(C(=O)NCc3ccc(-c4nncnn4)cc3)n2)c1]

To a stirred solution of 6-[2-(3,5-dimethoxyphenyl)-4-methyl-phenyl]sulfanylpyridine-2-carboxylic acid (150 mg, 0.39 mmol) and [4-(1,2,4,5-tetrazin-3-yl)phenyl]methanamine hydrochloride salt (90 mg, 0.40 mmol, Click Chemistry Tools, CAS #1416711-59-5) in dichloromethane (4 mL) is added HATU (190 mg, 0.49 mmol) and N, N-diisopropylethylamine (0.14 mL, 0.80 mmol). The mixture is stirred at room temperature overnight. The reaction mixture is concentrated to partial volume under reduced pressure, diluted with 30 mL ethyl acetate, and washed sequentially with 0.5M aqueous KHCO₃ (twice) and saturated aqueous NaCl (once), dried over Na₂SO₄, filtered, and concentrated under reduced pressure. The resulting residue is purified by column chromatography over silica, eluting with a gradient of 0-90% ethyl acetate in hexanes, to afford 6-[2-(3,5-dimethoxyphenyl)-4-methyl-phenyl]sulfanyl-N-[[4-(1,2,4,5-tetrazin-3-yl)phenyl]methyl]pyridine-2-carboxamide (180 mg, 85% yield) as a purple foamy solid. ES/MS (m/z): 551 (M+H).

To a stirred solution of 6-[2-(3,5-dimethoxyphenyl)-4-methyl-phenyl]sulfanyl-N-[[4-(1,2,4,5-tetrazin-3-yl)phenyl]methyl]pyridine-2-carboxamide (180 mg, 0.33 mmol) in dichloromethane (10 mL) is added 3-chloroperoxybenzoic acid (77% pure, 73 mg, 0.33 mmol). The resulting mixture is stirred at room temperature for 2-3 hrs. The mixture is purified by column chromatography over silica, eluting with a gradient of 0-100% ethyl acetate in hexanes, to afford the title compound (167 mg, 99% yield) as a purple foamy solid. ES/MS (m/z): 567 (M+H).

### Preparation 6

### 6-(2,4-dimethoxy-8-methyl-dibenzothiophen-5-ium-5-yl)-N-[[4-(1,2,4,5-tetrazin-3-yl)phenyl]methyl]pyridine-2-carboxamide trifluoromethanesulfonate

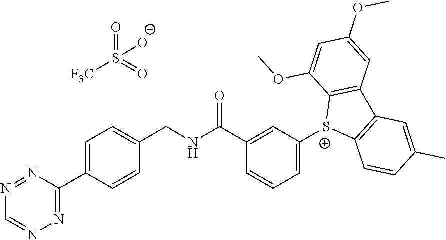 [SMILES: COc1cc(OC)c2c(c1)c1cc(C)ccc1[s+]2-c1cccc(C(=O)NCc2ccc(-c3nncnn3)cc2)c1 ; O=S(=O)([O-])C(F)(F)F]

To a stirred suspension of 6-[2-(3,5-dimethoxyphenyl)-4-methyl-phenyl]sulfinyl-N-[[4-(1,2,4,5-tetrazin-3-yl)phenyl]methyl]pyridine-2-carboxamide (88 mg, 0.16 mmol) and polymer supported-triethylamine (0.4 g, 1 mmol, 3.2 mmol/g) in dichloromethane (8 mL) at 0° C. is added a 1M solution of trifluoromethanesulfonic anhydride (0.42 mL, 0.42 mmol) in dichloromethane. The mixture is stirred at room temperature for 30 min and quenched with a drop of water. The resulting suspension is filtered, washed with dichloromethane, and concentrated under reduced pressure. The resulting residue is purified by column chromatography over silica, eluting with a gradient of 0-15% methanol in dichloromethane, to afford the title compound (91 mg, 81% yield) as a purple solid. ¹H NMR (400 MHz, DMSO-d₆) δ ppm: 2.49 (s, 3H), 4.01-4.00 (m, 6H), 4.75-4.63 (m, 2H), 5.75 (s, 1H), 7.01 (d, J=2.1 Hz, 1H), 7.62-7.59 (m, 3H), 7.76-7.72 (m, 2H), 8.23 (t, J=7.8 Hz, 1H), 8.30 (dd, J=0.9, 7.8 Hz, 1H), 8.37-8.34 (m, 1H), 8.44 (d, J=8.3 Hz, 1H), 8.55-8.52 (m, 2H), 9.12-9.08 (m, 1H), 10.61 (s, 1H). ¹⁹F NMR (376 MHz, DMSO-d₆) δ ppm:−77.7. ES/MS (m/z): 549 (M+).

### EXAMPLE 4

[¹⁸F]N-(4-(1,2,4,5-tetrazin-3-yl)benzyl)-6-fluoropicolinamide

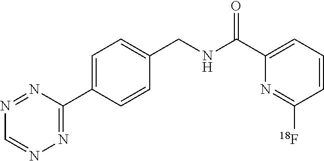 [SMILES: O=C(NCc1ccc(-c2nncnn2)cc1)c1cccc([18F])n1]

Typical radiochemical yield of [¹⁸F]N-(4-(1,2,4,5-tetrazin-3-yl)benzyl)-6-fluoropicolinamide radiosynthesis is 18.5±4.6% (N=6) using 0.675 to 1.327 Ci starting activity with synthesis time of 45-50 minutes.

[¹⁸F] Fluoride activity (0.195 to 1.475 Ci) is retained on a Sep-Pak Accell Plus QMA Plus Light Cartridge (130 mg, 37-55 μm, Waters Part #WAT023525; preconditioned with 5 mL of water for injection) and is eluted into a TRACERlab FXF-N reaction vessel using 0.8 mL of an aqueous tetraethylammonium bicarbonate solution [tetraethylammonium bicarbonate (3.5 mg) in water (0.2 mL) and acetonitrile (0.6 mL)]. The eluted activity is heated to 70° C. and dried under a compressed nitrogen gas purge and vacuum for 5 min. The temperature is increased to 100° C. and held for 5 min under vacuum, yielding [¹⁸F]tetraethylammonium fluoride. 1 mL of anhydrous DMSO is added to the reactor and the reactor is cooled to 35° C. The [¹⁸F]tetraethylammonium fluoride solution is transferred in the TRACERlab injection vial (RV2) containing a solution of 6-(2,4-dimethoxy-8-methyl-dibenzothiophen-5-ium-5-yl)-N-[[4-(1,2,4,5-tetrazin-3-yl)phenyl]methyl]pyridine-2-carboxamide trifluoromethanesulfonate (1 mg, 1.43 μmol) in anhydrous DMSO (0.2 mL) and kept at room temperature for 5 min. The mixure is diluted with 3.5 mL of 0.1% (v/v) trifluoroacetic acid in water and the resulting crude reaction mixture loaded onto a semi-preparative HPLC column for purification (conditions listed in FIG. 3). The HPLC fraction containing the purified [¹⁸F]N-(4-(1,2,4,5-tetrazin-3-yl)benzyl)-6-fluoropicolinamide (FIG. 3) is collected into a vial containing 40 mL of water and loaded onto a Sep-Pak® Light 1cc Vac C18 cartridge (50 mg, 55-105 μm, Waters Part #WAT054955; preconditioned with 5 mL of EtOH and 5 mL of water). The retained [¹⁸F]N-(4-(1,2,4,5-tetrazin-3-yl)benzyl)-6-fluoropicolinamide is eluted using 0.5 mL of EtOH and reconstituted into a formulation of 5 mL of 10% (v/v) EtOH in phosphate-buffered saline. The amount of [¹⁸F]N-(4-(1,2,4,5-tetrazin-3-yl)benzyl)-6-fluoropicolinamide obtained ranged from 0.0085 to 0.066 Ci. Representative analytical HPLC of formulated [18]N-(4-(1,2,4,5-tetrazin-3-yl)benzyl)-6-fluoropicolinamide are shown in FIG. 4.

### COMPARATITVE REFERENCE: EXAMPLE 5

### N-(4-(1,2,4,5-tetrazin-3-yl)benzyl)-6-fluoronicotinamide

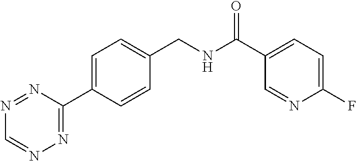 [SMILES: O=C(NCc1ccc(-c2nncnn2)cc1)c1ccc(F)nc1]

To a stirred solution of [4-(1,2,4,5-tetrazin-3-yl)phenyl]methanamine hydrochloride (60 mg, 0.27 mmol, Click Chemistry Tools, CAS #1416711-59-5) and 6-fluoropyridine-3-carboxylic acid (46 mg, 0.33 mmol) in dichloromethane (4 mL) is added HATU (135 mg, 0.35 mmol) and N,N-diisopropylethylamine (0.1 mL, 0.57 mmol). The reaction mixture is stirred at room temperature overnight. The reaction mixture is concentrated and diluted with ethyl acetate, the organic layer is washed sequentially with saturated aqueous NaHCO₃, saturated aqueous NaCl, dried over Na₂SO₄, filtered, and concentrated under reduced pressure. The resulting residue is purified by column chromatography over silica, eluting with a gradient of 0-50% dichloromethane in ethyl acetate, to give the title compound as a purple solid (18 mg, 0.06 mmol).

¹H NMR (400.13 MHz, CDCl₃): 4.81 (d, J=5.9 Hz, 2H), 6.61-6.56 (m, 1H), 7.06 (dd, J=2.1, 8.4 Hz, 1H), 7.62 (d, J=8.1 Hz, 2H), 8.35-8.30 (m, 1H), 8.69-8.64 (m, 3H), 10.24 (s, 1H), ¹⁹F NMR (376.45 MHz, CDCl₃) δ ppm:−62.8. ES/MS (m/z): 311 [M+H)]⁺.

### Preparation 7

### 6-[2-(3,5-dimethoxyphenyl)-4-methyl-phenyl]sulfanylpyridine-3-carboxylic acid

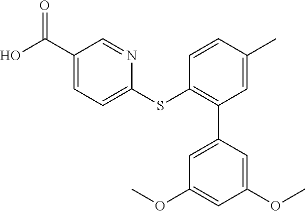 [SMILES: COc1cc(OC)cc(-c2cc(C)ccc2Sc2ccc(C(=O)O)cn2)c1]

A solution of ethyl 6-chloropyridine-3-carboxylate (280 mg, 1.51 mmol) and 2-ethylhexyl 3-[2-(3,5-dimethoxyphenyl)-4-methyl-phenyl]sulfanylpropanoate (740 mg, 1.66 mmol) in 1,4-dixoane (10 mL) is added 1M solution of potassium tert-butoxide in THF (1.8 mL, 1.8 mmol). The solution is heated to 80° C. for 4-5 hours, then stirred at room temperature overnight. 2 M aqueous solution of lithium hydroxide (10 mL, 20 mmol) is added, and the mixture stirred at 80° C. for 2-3 hours. The solution is cooled to room temperature and acidified with 2N HCl and extracted with ethyl acetate. The organic layer is then dried over Na₂SO₄, filtered and concentrated. The residue is purified by silica gel column (0-80% hexanes/ethyl acetate) to afford the title compound (527 mg, 1.31 mmol) as an off-white solid. ES/MS (m/z): 382 [M+H]+.

### Preparation 8

### N-(4-(1,2,4,5-tetrazin-3-yl)benzyl)-6-((3′,5′-dimethoxy-5-methyl-[1,1′-biphenyl]-2-yl)sulfinyl)nicotinamide

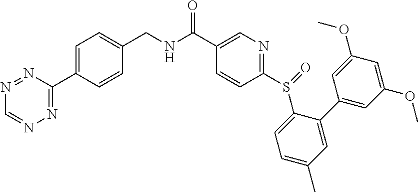 [SMILES: COc1cc(OC)cc(-c2cc(C)ccc2S(=O)c2ccc(C(=O)NCc3ccc(-c4nncnn4)cc3)cn2)c1]

To a stirred mixture of 6-[2-(3,5-dimethoxyphenyl)-4-methyl-phenyl]sulfanylpyridine-3-carboxylic acid (94 mg, 0.25 mmol) and [4-(1,2,4,5-tetrazin-3-yl)phenyl]methanamine hydrochloride (55 mg, 0.25 mmol) and HATU (115 mg, 0.30 mmol) in dichloromethane (2 mL) is added N,N-diisopropylethylamine (0.07 mL, 0.4 mmol). The reaction mixture is stirred at room temperature overnight. The resulting residue is diluted with dichloromethane and washed sequentially with saturated aqueous NaHCO₃ and saturated aqueous NaCl. The organic extracts are dried over Na₂SO₄, filtered, and concentrated under reduced pressure. The resulting residue is purified by chromatography on silica gel (0-80% hexanes/ethyl acetate) to afford N-(4-(1,2,4,5-tetrazin-3-yl)benzyl)-6-((3′,5′-dimethoxy-5-methyl-[1,1′-biphenyl]-2-yl)thio)nicotinamide (70 mg, 0.13 mmol) as a pink foam.

To a solution of N-(4-(1,2,4,5-tetrazin-3-yl)benzyl)-6-((3′,5′-dimethoxy-5-methyl-[1,1′-biphenyl]-2-yl)thio)nicotinamide (70 mg, 0.13 mmol) in dichloromethane (4 mL) is added 3-chloroperoxybenzoic acid (20 mg, 0.09 mmol, 77% pure). The mixture is stirred at room temperature for 2-3 hrs. The mixture is then purified by silica gel column (12g, 0-100% hexanes/ethyl acetate) to afford the title compound (24 mg) as a pink foam: ES/MS (m/z): 567 [M+H]⁺.

### Preparation 9

### 5-(5-((4-(1,2,4,5-tetrazin-3-yl)benzyl)carbamoyl)pyridin-2-yl)-2,4-dimethoxy-8-methyl-5H-dibenzo[b,d]thiophen-5-ium trifluoromethanesulfonate

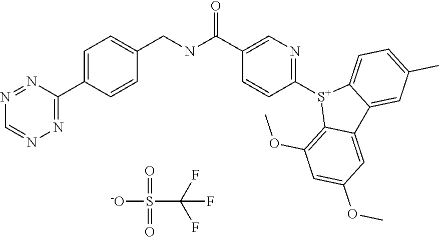

To a stirred slurry of N-(4-(1,2,4,5-tetrazin-3-yl)benzyl)-6-((3′,5′-dimethoxy-5-methyl-[1,1′-biphenyl]-2-yl)sulfinyl)nicotinamide (23 mg, 0.04 mmol) and diethylaminopolystyrene (3.2 mmol/g, 70 mg, 0.22 mmol) in dichloromethane (3 mL) cooled to 0° C. is added a 1M solution of trifluoromethanesulfonic anhydride in dichloromethane (130 μL, 0.13 mmol). The reaction mixture is warmed to room temperature, stirred for 30 minutes. The reaction is quenched by adding a drop of water. The mixture is filtered, and solids are washed with dichloromethane. The combined filtrates are concentrated under reduced pressure and the resulting residue purified by chromatography on silica gel, using a gradient of 0-20% methanol in dichloromethane, to afford the title compound (12 mg, 0.017 mmol, 41% yield) as a purple solid: ¹H NMR (400.13 MHz, DMSO-d₆) δ ppm: 2.55 (s, 3H), 3.95 (s, 3H), 4.00 (s, 3H), 4.64-4.62 (m, 2H), 6.92 (d, J=2.1 Hz, 1H), 7.62-7.57 (m, 3H), 7.69 (d, J=2.1 Hz, 8.26 (d, J=8.2 Hz, 1H), 8.39-8.37 (m, 2H), 8.48-8.44 (m, 2H), 8.54-8.52 (m, 1H), 8.92-8.91 (m, 1H), 9.48-9.44 (m, 1H), 10.58 (s, 1H), ¹⁹F NMR (376.45 MHz, DMSO-d₆) δ ppm:−77.7. ES/MS (m/z): 549 [M⁺].

### COMPARATITVE REFERENCE EXAMPLE 6

### [¹⁸]FN-(4-(1,2,4,5-tetrazin-3-yl)benzyl)-6-fluoronicotinamide

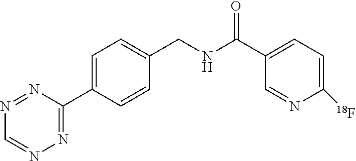 [SMILES: O=C(NCc1ccc(-c2nncnn2)cc1)c1ccc([18F])nc1]

Typical radiochemical yield of [¹⁸F]N-(4-(1,2,4,5-tetrazin-3-yl)benzyl)-6-fluoronicotinamide radiosynthesis is 20±2.18% (N=3) using 0.318 to 0.615 Ci starting activity with synthesis time of 50 minutes.

[¹⁸F] Fluoride activity (0.318 to 0.615 Ci) is retained on a Sep-Pak Accell Plus QMA Plus Light Cartridge (130 mg, 37-55 μm, Waters Part #WAT023525); and eluted into a TRACERlab FX_{F-N} reaction vessel using 0.8 mL of an aqueous tetraethylammonium bicarbonate solution [tetraethylammonium bicarbonate (3.6 mg) in water (0.2 mL) and acetonitrile (0.6 mL)]. The eluted activity is heated to 70° C. and dried under a compressed nitrogen gas purge and vacuum for 5 minutes. The temperature is then raised to 100° C. and held for 5 minutes under vacuum yielding tetraethylammonium [¹⁸F]fluoride. The reactor is then cooled to 30° C. and 1.2 mL of anhydrous DMSO is added to the reactor. The tetraethylammonium [¹⁸F]fluoride solution is transferred in the TRACERlab injection vial (RV2) containing precursor solution of 5-(5-((4-(1,2,4,5-tetrazin-3-yl)benzyl)carbamoyl)pyridin-2-yl)-2,4-dimethoxy-8-methyl-5H-dibenzo[b,d]thiophen-5-ium trifluoromethanesulfonate [(1 mg, 1.83 μmol) in 0.3 mL of anhydrous DMSO] and kept at room temperature for 2 minutes. The mixture is then diluted with 3.5 mL of 1% (v/v) TFA in water and the resulting crude reaction mixture loaded onto a semi-preparative HPLC column for purification (conditions listed in FIG. 5). The HPLC fraction containing the purified [¹⁸F]N-(4-(1,2,4,5-tetrazin-3-yl)benzyl)-6-fluoronicotinamide (FIG. 5) is collected into a vial containing 40 mL of 0.02% (v/v) TFA in water and loaded onto a Sep-Pak® Light C18 cartridge (130 mg, 55-105 μm, Waters Part #WAT023501).

The retained [¹⁸F]N-(4-(1,2,4,5-tetrazin-3-yl)benzyl)-6-fluoronicotinamide is washed with 5 mL of 0.02% (v/v) TFA in water, eluted using 1 mL of EtOH and reconstituted into a formulation of 10 mL of 10% (v/v) EtOH in PBS. The amount of [¹⁸F]N-(4-(1,2,4,5-tetrazin-3-yl)benzyl)-6-fluoronicotinamide obtained ranged from 0.046 to 0.125 Ci. Representative analytical HPLC chromatograms of formulated [¹⁸F]N-(4-(1,2,4,5-tetrazin-3-yl)benzyl)-6-fluoronicotinamide are shown in FIG. 6.

### EXAMPLE 2

### PET-CT imaging of [¹⁸F]N-(4-(1,2,4,5-tetrazin-3-yl)benzyl)-2-fluoronicotinamide

A Siemens Inveon® Multimodality Scanner (Siemens, Germany) is used for micro PET-CT imaging. Male CD-1 (6-week-old, ˜30 g) mice are anesthetized with 3% isoflurane/97% oxygen and placed on the bed of the scanner. The mice are administered [¹⁸F]N-(4-(1,2,4,5-tetrazin-3-yl)benzyl)-2-fluoronicotinamide via a bolus intravenous tail vein injection (˜350 ρCi in a total volume of 150 μL saline). A total of four dynamic PET scans are conducted, followed by a short high-resolution CT scan for anatomical registration. PET images are generated for each minute of the acquisition time. Uptake of the tracer in the brain, muscle, and bone are determined by visually drawing regions of interest (ROIs) based on the fused PET/CT images and the corresponding activity values are determined using the Inveon® Research Workplace software. All values are represented as % injected dose per gram (% ID/g).

Analysis of the four 60-minute PET scans indicate [¹⁸F]N-(4-(1,2,4,5-tetrazin-3-yl)benzyl)-2-fluoronicotinamide readily crosses the blood-brain-barrier. Peak brain uptake of 3.3% ID/g is observed at 4.5 min post injection followed by a steady clearance of tracer to 1.2% ID/g at 59.5 min. Uptake of [¹⁸F]N-(4-(1,2,4,5-tetrazin-3-yl)benzyl)-2-fluoronicotinamide is also observed throughout the peripheral of the body in organs such as the liver, kidneys and heart. Bone uptake throughout the scan period remained low, consistent with an absence of defluorination. Time activity curves are generated for [¹⁸F]N-(4-(1,2,4,5-tetrazin-3-yl)benzyl)-2-fluoronicotinamide in CD-1 male mice (n=4). Data is shown in tabular format (Table 1) below.

Table 1 represents the 60 min PET time activity results for [¹⁸F]N-(4-(1,2,4,5-tetrazin-3-yl)benzyl)-2-fluoronicotinamide (brain, muscle, and bone, N=4).

TABLE 1
60-min PET time activity table for [¹⁸F]N-(4-(1,2,4,5-tetrazin-
3-yl)benzyl)-2-fluoronicotinamide (Example 2) (brain, muscle, & bone)

| | Brain | | | Muscle | | | Bone | | |
|---|---|---|---|---|---|---|---|---|---|
| Time | Mean | | | Mean | | | Mean | | |
| (min) | (% ID/g) | SEM | N | (% ID/g) | SEM | N | (% ID/g) | SEM | N |
| 0 | 0.00 | 0.00 | 4 | 0.00 | 0.00 | 4 | 0.00 | 0.00 | 4 |
| 0.5 | 0.00 | 0.00 | 4 | 0.00 | 0.00 | 4 | 0.00 | 0.00 | 4 |
| 1.5 | 0.64 | 0.24 | 4 | 0.16 | 0.10 | 4 | 0.19 | 0.10 | 4 |
| 2.5 | 2.74 | 0.93 | 4 | 0.76 | 0.31 | 4 | 0.95 | 0.37 | 4 |
| 3.5 | 2.47 | 0.80 | 4 | 0.79 | 0.32 | 4 | 1.06 | 0.39 | 4 |
| 4.5 | 3.27 | 0.35 | 4 | 1.13 | 0.20 | 4 | 1.32 | 0.14 | 4 |
| 5.5 | 3.05 | 0.34 | 4 | 1.21 | 0.18 | 4 | 1.37 | 0.14 | 4 |
| 6.5 | 2.95 | 0.28 | 4 | 1.33 | 0.18 | 4 | 1.42 | 0.15 | 4 |
| 7.5 | 2.74 | 0.27 | 4 | 1.36 | 0.19 | 4 | 1.44 | 0.14 | 4 |
| 8.5 | 2.57 | 0.30 | 4 | 1.44 | 0.17 | 4 | 1.52 | 0.11 | 4 |
| 9.5 | 2.42 | 0.29 | 4 | 1.51 | 0.17 | 4 | 1.56 | 0.10 | 4 |
| 10.5 | 2.29 | 0.28 | 4 | 1.53 | 0.18 | 4 | 1.58 | 0.08 | 4 |
| 11.5 | 2.18 | 0.26 | 4 | 1.57 | 0.16 | 4 | 1.54 | 0.10 | 4 |
| 12.5 | 2.07 | 0.24 | 4 | 1.60 | 0.13 | 4 | 1.58 | 0.12 | 4 |
| 13.5 | 1.97 | 0.22 | 4 | 1.58 | 0.17 | 4 | 1.63 | 0.09 | 4 |
| 14.5 | 1.89 | 0.20 | 4 | 1.58 | 0.15 | 4 | 1.58 | 0.07 | 4 |
| 15.5 | 1.82 | 0.20 | 4 | 1.67 | 0.14 | 4 | 1.59 | 0.08 | 4 |
| 16.5 | 1.75 | 0.19 | 4 | 1.64 | 0.15 | 4 | 1.64 | 0.08 | 4 |
| 17.5 | 1.70 | 0.17 | 4 | 1.65 | 0.11 | 4 | 1.65 | 0.07 | 4 |
| 18.5 | 1.65 | 0.16 | 4 | 1.59 | 0.12 | 4 | 1.64 | 0.06 | 4 |
| 19.5 | 1.60 | 0.15 | 4 | 1.64 | 0.12 | 4 | 1.65 | 0.09 | 4 |
| 20.5 | 1.56 | 0.14 | 4 | 1.63 | 0.11 | 4 | 1.62 | 0.04 | 4 |
| 21.5 | 1.51 | 0.13 | 4 | 1.67 | 0.10 | 4 | 1.61 | 0.09 | 4 |
| 22.5 | 1.47 | 0.12 | 4 | 1.68 | 0.12 | 4 | 1.59 | 0.02 | 4 |
| 23.5 | 1.45 | 0.11 | 4 | 1.66 | 0.13 | 4 | 1.63 | 0.04 | 4 |
| 24.5 | 1.42 | 0.11 | 4 | 1.69 | 0.10 | 4 | 1.63 | 0.08 | 4 |
| 25.5 | 1.39 | 0.09 | 4 | 1.64 | 0.13 | 4 | 1.62 | 0.05 | 4 |
| 26.5 | 1.37 | 0.09 | 4 | 1.62 | 0.13 | 4 | 1.57 | 0.05 | 4 |
| 27.5 | 1.36 | 0.08 | 4 | 1.69 | 0.11 | 4 | 1.62 | 0.05 | 4 |
| 28.5 | 1.33 | 0.09 | 4 | 1.65 | 0.13 | 4 | 1.61 | 0.03 | 4 |
| 29.5 | 1.32 | 0.08 | 4 | 1.68 | 0.10 | 4 | 1.66 | 0.05 | 4 |
| 30.5 | 1.29 | 0.07 | 4 | 1.72 | 0.11 | 4 | 1.64 | 0.02 | 4 |
| 31.5 | 1.29 | 0.07 | 4 | 1.64 | 0.11 | 4 | 1.58 | 0.04 | 4 |
| 32.5 | 1.27 | 0.07 | 4 | 1.66 | 0.08 | 4 | 1.64 | 0.03 | 4 |
| 33.5 | 1.26 | 0.06 | 4 | 1.69 | 0.08 | 4 | 1.55 | 0.03 | 4 |
| 34.5 | 1.25 | 0.06 | 4 | 1.62 | 0.09 | 4 | 1.67 | 0.03 | 4 |
| 35.5 | 1.25 | 0.06 | 4 | 1.65 | 0.15 | 4 | 1.57 | 0.05 | 4 |
| 36.5 | 1.24 | 0.06 | 4 | 1.70 | 0.11 | 4 | 1.61 | 0.07 | 4 |
| 37.5 | 1.22 | 0.05 | 4 | 1.66 | 0.09 | 4 | 1.58 | 0.02 | 4 |
| 38.5 | 1.21 | 0.06 | 4 | 1.62 | 0.09 | 4 | 1.61 | 0.05 | 4 |
| 39.5 | 1.21 | 0.05 | 4 | 1.67 | 0.14 | 4 | 1.66 | 0.03 | 4 |
| 40.5 | 1.21 | 0.05 | 4 | 1.65 | 0.11 | 4 | 1.60 | 0.03 | 4 |
| 41.5 | 1.20 | 0.05 | 4 | 1.70 | 0.10 | 4 | 1.65 | 0.05 | 4 |
| 42.5 | 1.19 | 0.05 | 4 | 1.63 | 0.09 | 4 | 1.63 | 0.06 | 4 |
| 43.5 | 1.18 | 0.06 | 4 | 1.68 | 0.15 | 4 | 1.71 | 0.10 | 4 |
| 44.5 | 1.19 | 0.05 | 4 | 1.66 | 0.13 | 4 | 1.61 | 0.04 | 4 |
| 45.5 | 1.19 | 0.05 | 4 | 1.70 | 0.09 | 4 | 1.60 | 0.03 | 4 |
| 46.5 | 1.19 | 0.05 | 4 | 1.59 | 0.09 | 4 | 1.52 | 0.03 | 4 |
| 47.5 | 1.19 | 0.06 | 4 | 1.64 | 0.06 | 4 | 1.57 | 0.06 | 4 |
| 48.5 | 1.18 | 0.05 | 4 | 1.57 | 0.11 | 4 | 1.62 | 0.05 | 4 |
| 49.5 | 1.18 | 0.05 | 4 | 1.65 | 0.08 | 4 | 1.59 | 0.05 | 4 |
| 50.5 | 1.17 | 0.05 | 4 | 1.57 | 0.11 | 4 | 1.66 | 0.04 | 4 |
| 51.5 | 1.18 | 0.05 | 4 | 1.61 | 0.07 | 4 | 1.64 | 0.03 | 4 |
| 52.5 | 1.17 | 0.05 | 4 | 1.62 | 0.12 | 4 | 1.64 | 0.05 | 4 |
| 53.5 | 1.16 | 0.05 | 4 | 1.66 | 0.13 | 4 | 1.55 | 0.07 | 4 |
| 54.5 | 1.17 | 0.05 | 4 | 1.58 | 0.11 | 4 | 1.61 | 0.04 | 4 |
| 55.5 | 1.17 | 0.05 | 4 | 1.55 | 0.09 | 4 | 1.65 | 0.08 | 4 |
| 56.5 | 1.17 | 0.05 | 4 | 1.72 | 0.09 | 4 | 1.62 | 0.05 | 4 |
| 57.5 | 1.18 | 0.05 | 4 | 1.59 | 0.05 | 4 | 1.65 | 0.08 | 4 |
| 58.5 | 1.17 | 0.05 | 4 | 1.57 | 0.10 | 4 | 1.64 | 0.04 | 4 |
| 59.5 | 1.17 | 0.05 | 4 | 1.53 | 0.08 | 4 | 1.62 | 0.09 | 4 |

### EXAMPLE 4

### PET-CT imaging of [¹⁸F]N-(4-(1,2,4,5-tetrazin-3-yl)benzyl)-6-fluoropicolinamide

A Siemens Inveon® Multimodality Scanner (Siemens, Germany) is used for micro PET-CT imaging. Male CD-1 (6-week-old, ˜30 g) mice were anesthetized with 3% isoflurane/97% oxygen and placed on the bed of the scanner. The mice are administered [¹⁸F]N-(4-(1,2,4,5-tetrazin-3-yl)benzyl)-6-fluoropicolinamide via a bolus intravenous tail vein injection (˜300 ρCi in a total volume of 150 μL saline). A total of four dynamic PET scans are conducted, followed by a short high-resolution CT scan for anatomical registration. PET images are generated for each minute of the acquisition time. Uptake of the tracer in the brain, muscle, and bone are determined by visually drawing regions of interest (ROIs) based on the fused PET/CT images and the corresponding activity values are determined using the Inveon® Research Workplace software. All values are represented as % injected dose per gram (% ID/g).

Analysis of the four 60-minute PET scans indicate that [¹⁸F]N-(4-(1,2,4,5-tetrazin-3-yl)benzyl)-6-fluoropicolinamide readily crosses the blood-brain-barrier. Peak brain uptake of 4.3% ID/g is observed at 2.5 min post injection followed by a steady clearance of tracer to 1.1% ID/g at 59.5 min. Uptake of [¹⁸F]N-(4-(1,2,4,5-tetrazin-3-yl)benzyl)-6-fluoropicolinamide is also observed throughout the periphery of the body in organs such as the liver, kidneys and heart. Bone uptake throughout the scan period remained low, consistent with an absence of defluorination. Time activity curves are generated for [¹⁸F]N-(4-(1,2,4,5-tetrazin-3-yl)benzyl)-6-fluoropicolinamide in CD-1 male mice (n=4). Data is shown in tabular format (Table 2) below.

Table 2 represents the 60 minute PET time activity results for [¹⁸F]N-(4-(1,2,4,5-tetrazin-3-yl)benzyl)-6-fluoropicolinamide (brain, muscle, and bone, N=4).

TABLE 2
60-minute PET time activity table for [¹⁸F]N-
(4-(1,2,4,5-tetrazin-3-yl)benzyl)-6-fluoropicolinamide
(Example 4) (brain, muscle, and bone)

| | Brain | | | Muscle | | | Bone | | |
|---|---|---|---|---|---|---|---|---|---|
| Time | Mean | | | Mean | | | Mean | | |
| (min) | % ID/g | SEM | N | % ID/g | SEM | N | % ID/g | SEM | N |
| 0 | 0.00 | 0.00 | 4 | 0.00 | 0.00 | 4 | 0.00 | 0.00 | 4 |
| 0.5 | 0.00 | 0.00 | 4 | 0.00 | 0.00 | 4 | 0.00 | 0.00 | 4 |
| 1.5 | 0.68 | 0.29 | 4 | 0.08 | 0.05 | 4 | 0.10 | 0.06 | 4 |
| 2.5 | 4.30 | 0.30 | 4 | 0.94 | 0.22 | 4 | 0.89 | 0.15 | 4 |
| 3.5 | 3.34 | 0.09 | 4 | 1.11 | 0.24 | 4 | 1.09 | 0.15 | 4 |
| 4.5 | 2.61 | 0.04 | 4 | 1.14 | 0.21 | 4 | 1.02 | 0.09 | 4 |
| 5.5 | 2.14 | 0.02 | 4 | 1.13 | 0.22 | 4 | 0.97 | 0.14 | 4 |
| 6.5 | 1.94 | 0.02 | 4 | 1.18 | 0.22 | 4 | 1.05 | 0.07 | 4 |
| 7.5 | 1.71 | 0.02 | 4 | 1.23 | 0.22 | 4 | 1.09 | 0.11 | 4 |
| 8.5 | 1.58 | 0.01 | 4 | 1.18 | 0.17 | 4 | 1.11 | 0.11 | 4 |
| 9.5 | 1.46 | 0.01 | 4 | 1.24 | 0.21 | 4 | 1.13 | 0.06 | 4 |
| 10.5 | 1.38 | 0.01 | 4 | 1.22 | 0.14 | 4 | 1.03 | 0.07 | 4 |
| 11.5 | 1.32 | 0.01 | 4 | 1.24 | 0.16 | 4 | 1.03 | 0.07 | 4 |
| 12.5 | 1.27 | 0.01 | 4 | 1.19 | 0.12 | 4 | 1.11 | 0.05 | 4 |
| 13.5 | 1.23 | 0.01 | 4 | 1.19 | 0.15 | 4 | 1.06 | 0.07 | 4 |
| 14.5 | 1.21 | 0.01 | 4 | 1.16 | 0.16 | 4 | 1.11 | 0.06 | 4 |
| 15.5 | 1.17 | 0.00 | 4 | 1.17 | 0.17 | 4 | 1.11 | 0.06 | 4 |
| 16.5 | 1.16 | 0.00 | 4 | 1.21 | 0.15 | 4 | 1.08 | 0.04 | 4 |
| 17.5 | 1.15 | 0.01 | 4 | 1.24 | 0.13 | 4 | 1.05 | 0.03 | 4 |
| 18.5 | 1.14 | 0.00 | 4 | 1.24 | 0.10 | 4 | 1.16 | 0.06 | 4 |
| 19.5 | 1.13 | 0.01 | 4 | 1.17 | 0.12 | 4 | 1.07 | 0.04 | 4 |
| 20.5 | 1.12 | 0.01 | 4 | 1.20 | 0.14 | 4 | 1.05 | 0.08 | 4 |
| 21.5 | 1.13 | 0.01 | 4 | 1.22 | 0.12 | 4 | 1.16 | 0.09 | 4 |
| 22.5 | 1.14 | 0.01 | 4 | 1.15 | 0.10 | 4 | 1.12 | 0.09 | 4 |
| 23.5 | 1.11 | 0.01 | 4 | 1.16 | 0.10 | 4 | 1.16 | 0.11 | 4 |
| 24.5 | 1.12 | 0.01 | 4 | 1.22 | 0.09 | 4 | 1.07 | 0.08 | 4 |
| 25.5 | 1.10 | 0.02 | 4 | 1.16 | 0.09 | 4 | 1.22 | 0.07 | 4 |
| 26.5 | 1.10 | 0.03 | 4 | 1.13 | 0.14 | 4 | 1.12 | 0.06 | 4 |
| 27.5 | 1.09 | 0.02 | 4 | 1.12 | 0.09 | 4 | 1.13 | 0.11 | 4 |
| 28.5 | 1.10 | 0.02 | 4 | 1.16 | 0.09 | 4 | 1.13 | 0.08 | 4 |
| 29.5 | 1.09 | 0.03 | 4 | 1.19 | 0.10 | 4 | 1.16 | 0.04 | 4 |
| 30.5 | 1.09 | 0.03 | 4 | 1.14 | 0.10 | 4 | 1.20 | 0.07 | 4 |
| 31.5 | 1.09 | 0.03 | 4 | 1.15 | 0.09 | 4 | 1.21 | 0.13 | 4 |
| 32.5 | 1.09 | 0.02 | 4 | 1.16 | 0.09 | 4 | 1.11 | 0.10 | 4 |
| 33.5 | 1.09 | 0.02 | 4 | 1.12 | 0.07 | 4 | 1.20 | 0.10 | 4 |
| 34.5 | 1.10 | 0.02 | 4 | 1.17 | 0.09 | 4 | 1.18 | 0.09 | 4 |
| 35.5 | 1.09 | 0.02 | 4 | 1.12 | 0.10 | 4 | 1.21 | 0.08 | 4 |
| 36.5 | 1.09 | 0.02 | 4 | 1.15 | 0.08 | 4 | 1.20 | 0.08 | 4 |
| 37.5 | 1.08 | 0.03 | 4 | 1.14 | 0.08 | 4 | 1.25 | 0.05 | 4 |
| 38.5 | 1.09 | 0.02 | 4 | 1.15 | 0.07 | 4 | 1.23 | 0.13 | 4 |
| 39.5 | 1.09 | 0.02 | 4 | 1.10 | 0.10 | 4 | 1.28 | 0.12 | 4 |
| 40.5 | 1.10 | 0.02 | 4 | 1.13 | 0.12 | 4 | 1.16 | 0.08 | 4 |
| 41.5 | 1.09 | 0.02 | 4 | 1.14 | 0.11 | 4 | 1.21 | 0.11 | 4 |
| 42.5 | 1.10 | 0.03 | 4 | 1.16 | 0.07 | 4 | 1.22 | 0.13 | 4 |
| 43.5 | 1.09 | 0.02 | 4 | 1.12 | 0.10 | 4 | 1.22 | 0.12 | 4 |
| 44.5 | 1.10 | 0.03 | 4 | 1.14 | 0.09 | 4 | 1.28 | 0.08 | 4 |
| 45.5 | 1.10 | 0.02 | 4 | 1.14 | 0.14 | 4 | 1.23 | 0.10 | 4 |
| 46.5 | 1.11 | 0.03 | 4 | 1.18 | 0.07 | 4 | 1.29 | 0.09 | 4 |
| 47.5 | 1.11 | 0.04 | 4 | 1.18 | 0.09 | 4 | 1.29 | 0.11 | 4 |
| 48.5 | 1.11 | 0.04 | 4 | 1.18 | 0.10 | 4 | 1.23 | 0.12 | 4 |
| 49.5 | 1.11 | 0.04 | 4 | 1.13 | 0.11 | 4 | 1.27 | 0.10 | 4 |
| 50.5 | 1.12 | 0.04 | 4 | 1.10 | 0.09 | 4 | 1.28 | 0.17 | 4 |
| 51.5 | 1.12 | 0.04 | 4 | 1.09 | 0.10 | 4 | 1.27 | 0.11 | 4 |
| 52.5 | 1.11 | 0.04 | 4 | 1.18 | 0.08 | 4 | 1.29 | 0.12 | 4 |
| 53.5 | 1.12 | 0.04 | 4 | 1.14 | 0.10 | 4 | 1.31 | 0.11 | 4 |
| 54.5 | 1.12 | 0.04 | 4 | 1.12 | 0.09 | 4 | 1.34 | 0.14 | 4 |
| 55.5 | 1.12 | 0.04 | 4 | 1.14 | 0.10 | 4 | 1.27 | 0.09 | 4 |
| 56.5 | 1.12 | 0.05 | 4 | 1.11 | 0.08 | 4 | 1.31 | 0.17 | 4 |
| 57.5 | 1.13 | 0.05 | 4 | 1.07 | 0.12 | 4 | 1.29 | 0.15 | 4 |
| 58.5 | 1.13 | 0.04 | 4 | 1.09 | 0.10 | 4 | 1.33 | 0.13 | 4 |
| 59.5 | 1.13 | 0.05 | 4 | 1.12 | 0.10 | 4 | 1.40 | 0.11 | 4 |

### COMPARATIVE EXAMPLE 6

### PET-CT imaging of [¹⁸F]N-(4-(1,2,4,5-tetrazin-3-yl)benzyl)-6-fluoronicotinamide

A Siemens Inveon® Multimodality Scanner (Siemens, Germany) is used for micro PET/CT imaging. Male CD-1 (6-week-old, 30-40 g) mice are anesthetized with 3% isoflurane/97% oxygen and placed on the bed of the scanner. The mice are administered [¹⁸F]N-(4-(1,2,4,5-tetrazin-3-yl)benzyl)-6-fluoronicotinamide via a bolus intravenous tail vein injection (˜300 ρCi in a total volume of 150 μL saline). A total of two dynamic PET scans are conducted, followed by a short high-resolution CT scan for anatomical registration. PET images are generated for each minute of the acquisition time. Uptake of the tracer in the brain, muscle, and bone are determined by visually drawing regions of interest (ROIs) based on the fused PET/CT images and the corresponding activity values are determined using the Inveon® Research Workplace software. All values are represented as % injected dose per gram (% ID/g).

Analysis of the two PET scans indicate that [¹⁸F]N-(4-(1,2,4,5-tetrazin-3-yl)benzyl)-6-fluoronicotinamide crosses the blood-brain-barrier. Peak brain uptake of 2.6% ID/g is observed at 2.5 min post injection followed by a steady clearance of tracer to 1.1% ID/g at 59.5 min. Uptake of [¹⁸F]N-(4-(1,2,4,5-tetrazin-3-yl)benzyl)-6-fluoronicotinamide is readily observed throughout the peripheral of the body in organs such as the liver, kidneys, intestines and heart. Bone uptake throughout the scan period remained low, consistent with an absence of defluorination. Time activity curves are generated for [¹⁸F]N-(4-(1,2,4,5-tetrazin-3-yl)benzyl)-6-fluoronicotinamide in CD-1 male mice (n=2). Data is shown in tabular format (Table 3) below.

Table 3 represents the 60 minute PET time activity results for [¹⁸F]N-(4-(1,2,4,5-tetrazin-3-yl)benzyl)-6-fluoronicotinamide (Brain, Muscle, & Bone, N=2)

TABLE 3
60 minute PET time activity table for [¹⁸F]N-
(4-(1,2,4,5-tetrazin-3-yl)benzyl)-6-fluoronicotinamide
(Comparative Example 6) (brain, muscle, & bone)

| | Brain | | | Muscle | | | Bone | | |
|---|---|---|---|---|---|---|---|---|---|
| Time | Mean | | | Mean | | | Mean | | |
| (min) | % ID/g | SEM | N | % ID/g | SEM | N | % ID/g | SEM | N |
| 0.0 | 0.00 | 0.00 | 2 | 0.00 | 0.00 | 2 | 0.00 | 0.00 | 2 |
| 0.5 | 0.00 | 0.00 | 2 | 0.00 | 0.00 | 2 | 0.00 | 0.00 | 2 |
| 1.5 | 0.52 | 0.52 | 2 | 0.07 | 0.07 | 2 | 0.09 | 0.09 | 2 |
| 2.5 | 2.57 | 0.24 | 2 | 0.56 | 0.11 | 2 | 0.83 | 0.13 | 2 |
| 3.5 | 2.45 | 0.07 | 2 | 0.67 | 0.09 | 2 | 0.95 | 0.19 | 2 |
| 4.5 | 2.29 | 0.09 | 2 | 0.73 | 0.02 | 2 | 0.85 | 0.07 | 2 |
| 5.5 | 2.20 | 0.08 | 2 | 0.72 | 0.02 | 2 | 0.86 | 0.06 | 2 |
| 6.5 | 2.23 | 0.08 | 2 | 0.80 | 0.00 | 2 | 0.90 | 0.13 | 2 |
| 7.5 | 2.15 | 0.08 | 2 | 0.84 | 0.01 | 2 | 0.90 | 0.10 | 2 |
| 8.5 | 2.10 | 0.09 | 2 | 0.84 | 0.03 | 2 | 0.90 | 0.10 | 2 |
| 9.5 | 2.00 | 0.12 | 2 | 0.78 | 0.03 | 2 | 0.81 | 0.15 | 2 |
| 10.5 | 1.95 | 0.12 | 2 | 0.79 | 0.01 | 2 | 0.90 | 0.06 | 2 |
| 11.5 | 1.89 | 0.12 | 2 | 0.84 | 0.01 | 2 | 0.92 | 0.14 | 2 |
| 12.5 | 1.86 | 0.13 | 2 | 0.84 | 0.02 | 2 | 0.94 | 0.20 | 2 |
| 13.5 | 1.83 | 0.13 | 2 | 0.88 | 0.02 | 2 | 0.97 | 0.11 | 2 |
| 14.5 | 1.78 | 0.12 | 2 | 0.88 | 0.04 | 2 | 0.98 | 0.00 | 2 |
| 15.5 | 1.76 | 0.14 | 2 | 0.89 | 0.04 | 2 | 0.98 | 0.12 | 2 |
| 16.5 | 1.72 | 0.12 | 2 | 0.93 | 0.07 | 2 | 0.83 | 0.11 | 2 |
| 17.5 | 1.69 | 0.15 | 2 | 0.91 | 0.04 | 2 | 1.00 | 0.18 | 2 |
| 18.5 | 1.65 | 0.13 | 2 | 0.89 | 0.00 | 2 | 0.97 | 0.08 | 2 |
| 19.5 | 1.63 | 0.14 | 2 | 0.90 | 0.03 | 2 | 0.93 | 0.17 | 2 |
| 20.5 | 1.61 | 0.15 | 2 | 0.95 | 0.07 | 2 | 1.01 | 0.03 | 2 |
| 21.5 | 1.58 | 0.15 | 2 | 0.98 | 0.07 | 2 | 0.91 | 0.25 | 2 |
| 22.5 | 1.56 | 0.15 | 2 | 0.95 | 0.03 | 2 | 1.02 | 0.10 | 2 |
| 23.5 | 1.55 | 0.17 | 2 | 1.00 | 0.09 | 2 | 1.04 | 0.06 | 2 |
| 24.5 | 1.51 | 0.15 | 2 | 0.98 | 0.05 | 2 | 1.11 | 0.14 | 2 |
| 25.5 | 1.49 | 0.15 | 2 | 0.99 | 0.09 | 2 | 0.93 | 0.06 | 2 |
| 26.5 | 1.46 | 0.14 | 2 | 0.95 | 0.05 | 2 | 0.86 | 0.09 | 2 |
| 27.5 | 1.45 | 0.15 | 2 | 0.94 | 0.03 | 2 | 0.94 | 0.08 | 2 |
| 28.5 | 1.42 | 0.14 | 2 | 1.00 | 0.06 | 2 | 1.00 | 0.08 | 2 |
| 29.5 | 1.40 | 0.15 | 2 | 0.97 | 0.05 | 2 | 1.05 | 0.03 | 2 |
| 30.5 | 1.38 | 0.16 | 2 | 0.99 | 0.05 | 2 | 0.95 | 0.14 | 2 |
| 31.5 | 1.38 | 0.15 | 2 | 0.95 | 0.01 | 2 | 1.04 | 0.05 | 2 |
| 32.5 | 1.36 | 0.15 | 2 | 0.97 | 0.04 | 2 | 1.04 | 0.09 | 2 |
| 33.5 | 1.36 | 0.14 | 2 | 1.00 | 0.07 | 2 | 1.01 | 0.01 | 2 |
| 34.5 | 1.34 | 0.16 | 2 | 1.04 | 0.06 | 2 | 1.06 | 0.15 | 2 |
| 35.5 | 1.33 | 0.15 | 2 | 1.04 | 0.08 | 2 | 0.97 | 0.10 | 2 |
| 36.5 | 1.30 | 0.16 | 2 | 1.04 | 0.07 | 2 | 1.01 | 0.03 | 2 |
| 37.5 | 1.31 | 0.15 | 2 | 1.03 | 0.05 | 2 | 0.99 | 0.10 | 2 |
| 38.5 | 1.29 | 0.15 | 2 | 1.03 | 0.05 | 2 | 1.12 | 0.01 | 2 |
| 39.5 | 1.26 | 0.16 | 2 | 1.03 | 0.05 | 2 | 1.13 | 0.05 | 2 |
| 40.5 | 1.25 | 0.14 | 2 | 1.05 | 0.07 | 2 | 1.07 | 0.17 | 2 |
| 41.5 | 1.26 | 0.17 | 2 | 1.08 | 0.08 | 2 | 1.06 | 0.10 | 2 |
| 42.5 | 1.23 | 0.15 | 2 | 1.06 | 0.05 | 2 | 1.00 | 0.01 | 2 |
| 43.5 | 1.23 | 0.17 | 2 | 1.07 | 0.06 | 2 | 1.09 | 0.02 | 2 |
| 44.5 | 1.21 | 0.15 | 2 | 1.02 | 0.03 | 2 | 0.97 | 0.08 | 2 |
| 45.5 | 1.21 | 0.15 | 2 | 1.10 | 0.07 | 2 | 1.09 | 0.00 | 2 |
| 46.5 | 1.19 | 0.15 | 2 | 1.06 | 0.05 | 2 | 1.04 | 0.16 | 2 |
| 47.5 | 1.19 | 0.16 | 2 | 1.07 | 0.05 | 2 | 1.07 | 0.01 | 2 |
| 48.5 | 1.18 | 0.17 | 2 | 1.06 | 0.02 | 2 | 1.06 | 0.11 | 2 |
| 49.5 | 1.18 | 0.16 | 2 | 1.06 | 0.00 | 2 | 1.03 | 0.09 | 2 |
| 50.5 | 1.17 | 0.16 | 2 | 1.11 | 0.06 | 2 | 1.27 | 0.09 | 2 |
| 51.5 | 1.16 | 0.16 | 2 | 1.09 | 0.05 | 2 | 1.15 | 0.03 | 2 |
| 52.5 | 1.15 | 0.17 | 2 | 1.10 | 0.05 | 2 | 1.19 | 0.14 | 2 |
| 53.5 | 1.14 | 0.16 | 2 | 1.10 | 0.04 | 2 | 1.08 | 0.09 | 2 |
| 54.5 | 1.13 | 0.15 | 2 | 1.10 | 0.02 | 2 | 1.11 | 0.14 | 2 |
| 55.5 | 1.09 | 0.13 | 2 | 1.11 | 0.05 | 2 | 1.13 | 0.01 | 2 |
| 56.5 | 1.09 | 0.13 | 2 | 1.05 | 0.02 | 2 | 1.13 | 0.13 | 2 |
| 57.5 | 1.09 | 0.13 | 2 | 1.13 | 0.08 | 2 | 1.09 | 0.01 | 2 |
| 58.5 | 1.08 | 0.13 | 2 | 1.05 | 0.01 | 2 | 1.03 | 0.09 | 2 |
| 59.5 | 1.09 | 0.13 | 2 | 1.09 | 0.03 | 2 | 1.07 | 0.00 | 2 |

The PET-CT data depicted in Tables 1 and 2 indicate that the ¹⁸F-labelled compounds of Example 2 and Example 4 cross the blood-brain barrier when tested in CD-1 mice. Peak brain uptake (% ID/g) is observed approximately 2 minutes post injection and then is followed by a steady clearance of tracer to baseline levels defined by uptake in the muscle at 60 minutes post injection. Bone uptake throughout the scan period remained low, consistent with the absence of in-vivo defluorination. These data indicate that the ¹⁸F-labelled compounds of Example 2 and Example 4 are useful as PET-CT imaging agents for CNS pretargeted imaging.

Analysis of the two PET scans indicate that [¹⁸F]N-(4-(1,2,4,5-tetrazin-3-yl)benzyl)-6-fluoronicotinamide (Comparative Example 6) crosses the blood-brain-barrier. Peak brain uptake of 2.6% ID/g is observed at 2.5 min post injection followed by a steady clearance of tracer to 1.1% ID/g at 59.5 min.

By comparison, dynamic PET imaging in CD-1 male mice of the [¹⁸F]N-(4-(1,2,4,5-tetrazin-3-yl)benzyl)-2-fluoronicotinamide (Example 2) and [¹⁸F]N-(4-(1,2,4,5-tetrazin-3-yl)benzyl)-6-fluoropicolinamide (Example 4) disclosed herein showed that these compounds readily cross the blood-brain-barrier and reaches a peak brain uptake of 3.3±0.4% ID/g and 4.3±0.3% ID/g, respectively. These compounds then displayed a steady clearance from the brain to near background levels (muscle) by 60 minutes post injection.

These agents possess higher brain penetration followed by rapid and complete washout from the brain and therefore provide a larger window to achieve higher signal to background ratios resulting in better image quality. Thus, we expect [¹⁸F]N-(4-(1,2,4,5-tetrazin-3-yl)benzyl)-2-fluoronicotinamide (Example 2) and [¹⁸F]N-(4-(1,2,4,5-tetrazin-3-yl)benzyl)-6-fluoropicolinamide (Example 4) disclosed herein to provide an advantage over [¹⁸F]N-(4-(1,2,4,5-tetrazin-3-yl)benzyl)-6-fluoronicotinamide (Comparative Example 6) for pretargeted CNS imaging.

In addition to the potential applications in pretargeted imaging studies, radiolabeled tetrazines have also been shown in multiple publications to be useful prosthetic groups for labeling biomolecules. Steven Liang and others have reported the sortase-mediated modification of camelid single-domain antibody fragments and subsequent radiolabeling with 18F-labeled tetrazines (Angew. Chem. Int. Ed. 2016, 55,528-533; WO 2017/059397 A1). Stina Syvänen and coworkers have reported the radiolabeling of bispecific antibodies via functionalization with trans-cyclooctene (TCO) groups and subsequent conjugation with 18F-labeled tetrazines performed at ambient temperature (ACS Chem. Neurosci. 2020, 11, 24, 4460-4468). Michael Zalutsky and coworkers have disclosed the radiolabeling of biomolecules such as nanobodies with radiolabeled tetrazines with both A1F chelate or fluoropyridine functional groups (Bioconjugate Chem. 2018, 29, 4090-4103; WO 2020/242948 A1).

Based on literature precedent, the tetrazines claimed herein would be expected to have utility as prosthetic groups for the labeling of biomolecules. The reactivity of tetrazines N-(4-(1,2,4,5-tetrazin-3-yl)benzyl)-2-fluoropicolinamide and N-(4-(1,2,4,5-tetrazin-3-yl)benzyl)-6-fluoropicolinamide with trans cyclooctene (TCO) conjugated to a biomolecule, Malat1 antisense oligonucleotide (ASO) (2019 World Molecular Imaging Congress Conference, Poster 139), was confirmed in both [¹⁹F] and [¹⁸F] experiments monitored with LC-MS and HPLC-radiodetection, respectively.

To a solution of 2.5 mg/mL (2.67 μM) Malat1 ASO-TCO in water, two equivalents tetrazine was added, and gently shaken at room temperature, protected from light, for 30 min. The reaction mixture was monitored by ES/MS on a Waters® Acquity UPLC system equipped with a Waters® Xevo QT of mass spectrometer. LC-MS conditions were as follows: column: Waters® ACQUITY PREMIER UPLC Oligonucleotide BEH C18 (130 Å. 1.7 μm, 2.1 mm×50 mm); wavelength 250-650 nm; gradient: initial hold at 5% B for 3.5 min, 5-75% B in 1.5 min, hold 75% B for 4 min, increase from 75-98% B in 0.5 min, hold 98% B for 1 min, and return to 5% B to reequilibrate; column temperature: 70° C. +/−5° C.; flow rate: 0.2 mL/min; solvent for A: 7 mM TEA and 100 mM HFIP in water; solvent for B; 7 mM TEA and 100 mM HFIP in 75% methanol/25% acetonitrile.

Negative mode mass spectra (500-3000 m/z) were summed from the peak of interest and processed in MaxEnt to produce zero-charge mass results. The mass result before and after reaction with each tetrazine (N-(4-(1,2,4,5-tetrazin-3-yl)benzyl)-2-fluoropicolinamide and N-(4-(1,2,4,5-tetrazin-3-yl)benzyl)-6-fluoropicolinamide) showed a difference of 282 Da (a shift from 7502 Da to 7784 Da) with no parent remaining indicating full reaction of Malat1 ASO-TCO with tetrazine (FIG. 7).

The concentration of tetrazine in solutions of [¹⁸F] N-(4-(1,2,4,5-tetrazin-3-yl)benzyl)-2-fluoropicolinamide and [¹⁸F] N-(4-(1,2,4,5-tetrazin-3-yl)benzyl)-6-fluoropicolinamide was calculated and 0.4 mL of each was reacted with 2 parts Malat1-ASO-TCO. Reactions were gently shaken at room temperature, protected from light, for 15 min prior to 100-fold dilution in water. Diluted samples were then analyzed on an Agilent 1290 series UPLC UV coupled with a BGO coincidence detector. HPLC conditions were as follows: column: Waters® ACQUITY PREMIER UPLC Oligonucleotide BEH C18 (130 Å, 1.7 μm, 2.1 mm×50 mm); gradient: initial hold at 5% B for 3 min, 5-75% B in 1.5 min, hold 75% B for 6 min, increase from 75-98% B in 1 min, hold 98% B for 1 min, and return to 5% B to re-equilibrate; column temperature: 70° C. +/−5° C.; flow rate: 0.25 mL/min; solvent for A: 7 mM TEA and 100 mM HFIP in water; solvent for B; 7 mM TEA and 100 mM HFIP in 75% methanol/25% acetonitrile.

Radiotraces for each tetrazine ([¹⁸F] N-(4-(1,2,4,5-tetrazin-3-yl)benzyl)-2-fluoropicolinamide and [¹⁸F] N-(4-(1,2,4,5-tetrazin-3-yl)benzyl)-6-fluoropicolinamide) before and after reaction with Malat1 ASO-TCO showed a shift from a single peak (RT=9 min), consisting of the unreacted tetrazine, to two peaks, with the earlier eluting peak (RT=3.4 min) representing the reacted species (FIG. 8).

Accordingly, the present embodiments include a method of radiolabelling a molecule is disclosed. Particularly, in the first step, a biomolecule is modified to attach or include a TCO moiety to form the biomolecule-TCO conjugate. (One exemplary way to do this is disclosed above in ACS Chem. Neurosci. 2020, 11, 24, 4460-4468). Then in the second step, the biomolecule-TCO conjugate would react with 18F-labelled tetrazines to form biomolecule-TCO-tetrazine-18F via a cycloaddition reaction.

Specifically, the method may include a method of radio-labelling a biomolecule comprising:
attaching a TCO-moeity to the biomolecule, thereby forming a biomolecule-TCO conjugate; and
reacting the biomolecule-TCO conjugate with a 18F-labelled tetrazine to form biomolecule-TCO-tetrazine-18F molecule, wherein the 18F-labelled tetrazine is selected from the group consisting of the Compound of Formula II and the Compound of Formula IV.

1.-17. (canceled)

18. A compound of the formula: 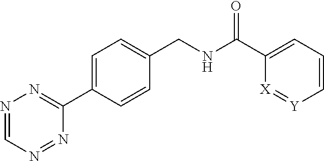
wherein:
X is N and Y is CF or C¹⁸F, or
Y is N and X is CF or C¹⁸F,
or a pharmaceutically acceptable salt thereof.

19. The compound of claim 18, which is

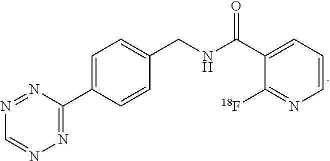 [SMILES: O=C(NCc1ccc(-c2nncnn2)cc1)c1cccnc1[18F]]

20. The compound of claim 18, which is

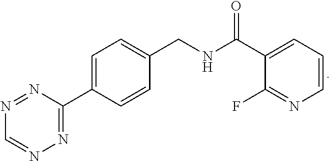 [SMILES: O=C(NCc1ccc(-c2nncnn2)cc1)c1cccnc1F]

21. The compound of claim 18, which is

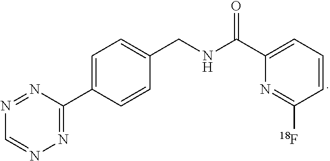 [SMILES: O=C(NCc1ccc(-c2nncnn2)cc1)c1cccc([18F])n1]

22. The compound of claim 18, which is

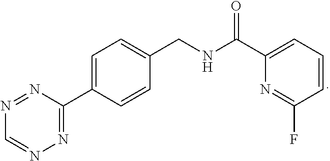 [SMILES: O=C(NCc1ccc(-c2nncnn2)cc1)c1cccc(F)n1]

23. A pharmaceutical composition comprising the compound or pharmaceutically acceptable salt thereof of claim 18, and one or more pharmaceutically acceptable carrier, diluent or excipient.

24. (canceled)

25. The pharmaceutical composition of claim 23, wherein the pharmaceutical composition does not include ascorbate or ascorbic acid.

26. A method of pretargeted imaging comprising:
a. introducing into a mammal a detectable quantity of the compound of claim 18, or pharmaceutically acceptable salt thereof, and
b. detecting said compound or pharmaceutically acceptable salt thereof in PET imaging, wherein the compound or pharmaceutically acceptable salt thereof is introduced after a biologics-TCO conjugate was introduced into the mammal.

27. A method of radio-labelling a biomolecule comprising:
attaching a TCO-moiety to the biomolecule, thereby forming a biomolecule-TCO conjugate; and
reacting the biomolecule-TCO conjugate with a 18F-labelled tetrazine to form biomolecule-TCO-tetrazine-18F molecule, wherein the 18F-labelled tetrazine is 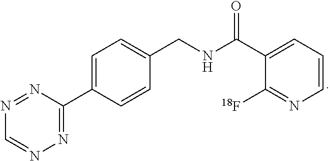 [SMILES: O=C(NCc1ccc(-c2nncnn2)cc1)c1cccnc1[18F]]
or

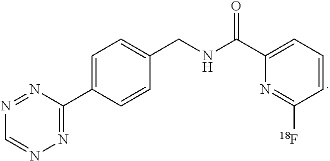 [SMILES: O=C(NCc1ccc(-c2nncnn2)cc1)c1cccc([18F])n1]

28. A compound of the formula

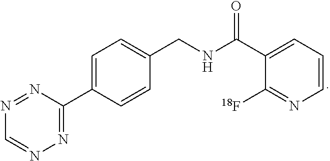 [SMILES: O=C(NCc1ccc(-c2nncnn2)cc1)c1cccnc1[18F]]

29. A compound of the formula

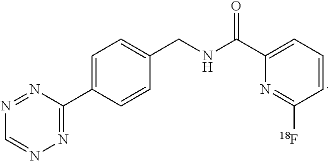 [SMILES: O=C(NCc1ccc(-c2nncnn2)cc1)c1cccc([18F])n1]

30. A compound selected from the group consisting of: 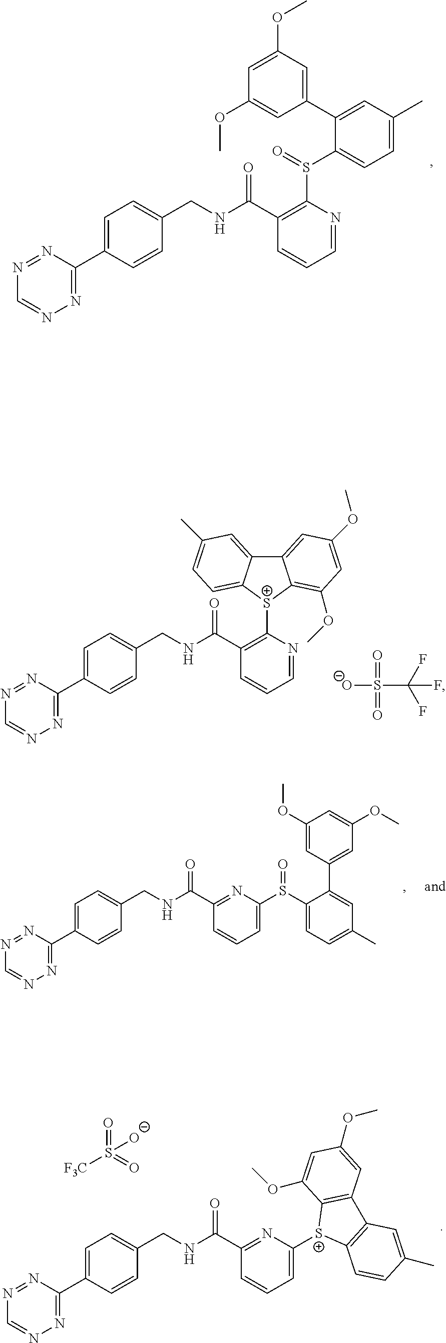 [SMILES: COc1cc(OC)cc(-c2cc(C)ccc2S(=O)c2ncccc2C(=O)NCc2ccc(-c3nncnn3)cc2)c1 ; COc1cc(OC)c2c(c1)c1cc(C)ccc1[s+]2-c1ncccc1C(=O)NCc1ccc(-c2nncnn2)cc1 ; O=S(=O)([O-])C(F)(F)F ; COc1cc(OC)cc(-c2cc(C)ccc2S(=O)c2cccc(C(=O)NCc3ccc(-c4nncnn4)cc3)n2)c1 ; COc1cc(OC)c2c(c1)c1cc(C)ccc1[s+]2-c1cccc(C(=O)NCc2ccc(-c3nncnn3)cc2)n1 ; O=S(=O)([O-])C(F)(F)F]
